(19) United States
(12) Patent Application Publication
(10) Pub. No.: US 2020/0281962 A1
(43) Pub. Date: Sep. 10, 2020

(54) NITRIC OXIDE RELEASING HIGH DENSITY LIPOPROTEIN-LIKE NANOPARTICLES (NO HDL NPS)

(71) Applicant: Northwestern University, Evanston, IL (US)

(72) Inventors: Jonathan RINK, Park Ridge, IL (US); Shad C. THAXTON, Chicago, IL (US); Melina KIBBE, Chicago, IL (US)

(73) Assignee: Northwestern University, Evanston, IL (US)

(21) Appl. No.: 16/063,546

(22) PCT Filed: Dec. 16, 2016
(86) PCT No.: PCT/US2016/067243

§ 371 Date: Jun. 18, 2018

Related U.S. Application Data
(60) Provisional application No. 62/269,859, filed on Dec. 18, 2015.

(51) Int. Cl. A61K 33/00; A61K 9/51; A61P 9/14

(52) U.S. Cl. CPC A61K 33/00; A61P 9/14; A61K 9/5169

(57) ABSTRACT

Nano structures having a core and a shell such as a lipid layer and optionally a lipoprotein which are useful for delivering nitric oxide are provided herein. Methods of treating disease using the nanostructures are also provided, including methods of treating vascular diseases, angiogenesis, ischemia-reperfusion, etc.

### RELATED APPLICATION

This application claims the benefit under 35 U.S.C. § 119(e) of U.S. provisional application No. 62/269,859, filed Dec. 18, 2015, which is incorporated by reference herein in its entirety.

### GOVERNMENT SUPPORT

This invention was made with government support under R01 HL116577 awarded by the National Institutes of Health. The government has certain rights in the invention.

### FIELD OF INVENTION

The present invention generally relates to nanoparticles designed to deliver nitric oxide (NO) as therapy for diseases.

### BACKGROUND

Narrowing of arteries, due to the proliferation and migration of the underlying muscle cells into the blood vessel, is a major complication of any therapeutic intervention taken to open a blocked artery, including balloon angioplasty. Currently, stents, including bare metal and drug loaded variants, are used to reduce the narrowing of the artery post procedure. However, narrowing can still occur with the bare metal stents, while the drug loaded stents have significant side effects associated with them and require patients to take blood thinners for the rest of their lives. Nitric oxide (NO), a highly reactive gas, has been demonstrated to have protective effects on blood vessels, significantly reducing narrowing after intervention as well as promoting the health of the cells lining the blood vessel. NO is extremely difficult to deliver, and currently there are no therapeutics that can deliver NO clinically. Attempts have been made to develop NO releasing nanoparticles/nanomaterials. In the prior attempts, limitations such as toxicity and instability of the nanomaterials in water/PBS in the materials being used (e.g. peptide amphiphiles, glass nanoparticles) prevented their application to biological systems.

### SUMMARY

The present invention relates to nanoparticles with reservoirs of nitric oxide and their use in the treatment of nitric oxide (NO)-mediated disorders and diseases. NO is a powerful vasodilator and second messenger involved in cell signaling. However, due to its high reactivity, NO has an extremely short half-life, rendering delivery problematic. In biological systems, S-nitrosylation of free thiols increases the half-life of NO. As is disclosed herein, an S-nitrosylated phospholipid was synthesized and characterized, and this molecule was incorporated into bio-inspired high-density lipoprotein-like nanoparticles (SNO HDL NPs).

As described herein, S-nitrosylation was achieved by adding sodium nitrite to a thiol-containing phospholipid under acidic conditions. This reaction led to rapid S-nitrosylation of the thiol-containing phospholipid (SNO-PL). The SNO-PL was used to synthesize SNO HDL NPs, whereby the amount of NO on the HDL NP was tailored. The SNO HDL NPs described herein retain NO for long periods of time. Furthermore, the SNO HDL NPs described herein reduce ischemia/reperfusion injury in a mouse kidney transplant model. The present disclosure details the synthesis of SNO-PL and the ability of SNO HDL NPs to deliver therapeutic quantities of NO to a cell and ameliorate NO-mediated disorders (e.g., ischemia/reperfusion injury).

According to one aspect, high density lipoprotein (HDL) nanoparticles that include nitric oxide (NO) are provided. In some embodiments, the HDL nanoparticle includes a core; a shell surrounding and attached to the nanostructure core, wherein the shell is comprised of apolipoprotein and reservoir molecules comprising NO.

In some embodiments, the reservoir molecule is a lipid. In some embodiments, the reservoir molecule is a phospholipid. In some embodiments, the reservoir molecule is a modified phospholipid. In some embodiments, the lipid contains an NO donating group. In some embodiments, the reservoir molecule is a S-Nitrosylated lipid. In certain embodiments, the reservoir molecule is S-Nitrosylated 1,2-dipalmitoyl-sn-glycero-3-phosphothioethanol (DPPTE).

In some embodiments, the apolipoprotein is apolipoprotein A-I (apoA-I).

In some embodiments, the core is an organic core. In some embodiments, the core is an inorganic core. In certain embodiments, the core is a gold core.

In some embodiments, the HDL nanoparticle has 60-250 fold excess lipid to gold core.

In some embodiments, the shell is a lipid shell. In some embodiments, the lipid shell is a lipid monolayer. In some embodiments, the lipid shell is a lipid bilayer.

In some embodiments, the reservoir molecule is not a lipid.

According to another aspect, methods for delivering NO to a subject are provided. In some embodiments, the method includes administering to the subject the HDL nanoparticle described herein to deliver NO to a cell in the subject.

According to another aspect, a structure that includes NO is provided: In some embodiments, the structure includes a nanostructure core, a shell surrounding and attached to the nanostructure core, wherein the shell includes reservoir molecules comprising a lipid and NO.

In some embodiments, the lipid is a modified lipid. In some embodiments, the lipid is a modified phospholipid. In some embodiments, the lipid contains an NO donating group. In some embodiments, the lipid is a S-Nitrosylated lipid. In certain embodiments, the lipid is S-Nitrosylated DPPTE.

In some embodiments, the structure further includes an apolipoprotein. In certain embodiments, the apolipoprotein is apoA-I.

In some embodiments, the core is an organic core. In some embodiments, the core is an inorganic core. In some embodiments, the core is a gold core.

In some embodiments, the structure has 60-250 fold excess lipid to gold core.

In some embodiments, the shell is a lipid shell. In some embodiments, the lipid shell is a lipid monolayer. In some embodiments, the lipid shell is a lipid bilayer.

In yet another aspect, methods for delivering NO to a subject are provided. In some embodiments, the method for delivering NO to a subject includes administering to the subject a structure described herein to deliver NO to a cell in the subject.

According to another aspect, methods for reducing cell migration are provided. In some embodiments, the method for reducing migration of a cell includes contacting the cell with an effective amount of the structure described herein to reduce migration of the cell relative to a cell without exposure to the structure.

In some embodiments, the cell is a neutrophil cell. In other embodiments, the cell is a muscle cell. In certain embodiments, the cell is an aortic smooth muscle cell. In some embodiments, the cell is an endothelial cell. In certain embodiments, the cell is an aortic endothelial cell.

In yet another aspect, methods for synthesizing a structure with a nitrosylated phospholipid are provided. In some embodiments, the method includes adding an equimolar amount of phospholipid and sodium nitrate under acidic conditions. The pH may be increased to neutralize the acidic conditions. The nitrosylated phospholipid is synthesized in an alcohol solution. The nitrosylated phospholipid is mixed with core apolipoprotein such that the structure can self-assemble.

In some embodiments, the acidic condition is an acidic pH. In some embodiments, the acidic pH is 3. In some embodiments, the alcohol solution is a 20% ethanol solution.

In yet another aspect, methods for treating a NO-mediated disorder includes administering to a subject having a NO-mediated disorder an effective amount of a nanostructure that includes a core, a shell surrounding and attached to the core, wherein the shell includes reservoir molecules that include NO to deliver NO to a cell of the subject and treat the NO-mediated disorder.

In some embodiments, the reservoir molecule is a lipid. In some embodiments, the reservoir molecule is a phospholipid. In some embodiments, the reservoir molecule is a modified phospholipid. In some embodiments, the lipid contains an NO donating group. In some embodiments, the reservoir molecule is a S-Nitrosylated lipid. In certain embodiments, the reservoir molecule is S-Nitrosylated DPPTE.

In some embodiments, the reservoir molecule is not a lipid.

In some embodiments, the core is an organic core. In some embodiments, the core is an inorganic core. In certain embodiments, the core is a gold core.

In some embodiments, the nanostructure has 60-250 fold excess lipid to gold core.

In some embodiments, the shell is a lipid shell. In some embodiments, the lipid shell is a lipid monolayer. In some embodiments, the lipid shell is a lipid bilayer.

In some embodiments, the NO-mediated disorder is angiogenesis. In some embodiments, the NO-mediated disorder is ischemia-reperfusion injury. In certain embodiments, the NO-mediated disorder is ischemia-reperfusion injury following organ transplantation.

In some embodiments, the organ is a kidney.

In some embodiments, the reservoir molecule includes a lipid.

In some embodiments, the nanostructure is a HDL nanoparticle.

According to another aspect, methods for transplanting a donor organ in a recipient subject are provided herein. In some embodiments, the method for transplanting a donor organ in a recipient subject includes harvesting a donor organ, contacting the donor organ with a nanostructure that includes a core, a shell surrounding and attached to the core, wherein the shell includes reservoir molecules that include NO; and transplanting the donor organ into a recipient subject, wherein the nanostructure reduces the risk of rejection of the donor organ relative to the risk of a donor organ transplanted without exposure to the nanostructure.

In some embodiments, the nanostructure is administered to the recipient subject after the donor organ is transplanted. In some embodiments, the nanostructure is administered to the donor before the donor organ is harvested. In some embodiments, the donor organ is contacted with the nanostructure after the donor organ is harvested and before the donor organ is transplanted.

In some embodiments, the nanostructure is administered to the recipient subject immediately after the donor organ is transplanted. In some embodiments, the method further includes administering to the recipient subject the nanostructure 24 hours after the donor organ is transplanted.

In some embodiments, the nanostructure reduces the levels of plasma creatine in the recipient subject relative to a recipient subject that received a transplanted donor organ without exposure to the nanostructure.

In some embodiments, the nanostructure reduces apoptosis of a cell in the donor organ relative to a cell in a donor organ transplanted without exposure to the nanostructure.

In some embodiments, the structure increases proliferation of a cell in the donor organ relative to a cell in a donor organ transplanted without exposure to the nanostructure.

In some embodiments, the transplanted organ is a kidney.

In some embodiments, the recipient subject is a mammal. In some embodiments, the recipient subject is a human.

In some embodiments, the donor subject is a mammal. In some embodiments, the donor subject is a human.

In some embodiments, the reservoir molecule is a lipid. In some embodiments, the reservoir molecule is a phospholipid. In some embodiments, the reservoir molecule is a modified phospholipid.

In some embodiments, the reservoir molecule contains an NO donating group.

In some embodiments, the reservoir molecule is a S-Nitrosylated lipid. In certain embodiments, the reservoir molecule is S-Nitrosylated DPPTE.

In some embodiments, the nanostructure further comprises an apolipoprotein. In certain embodiments, the apolipoprotein is apoA-I.

In some embodiments, the core is an organic core. In some embodiments, the core is an inorganic core. In some embodiments, the core is a gold core.

In some embodiments, the nanostructure has 60-250 fold excess lipid to gold core.

In some embodiments, the shell is a lipid shell. In some embodiments, the lipid shell is a lipid monolayer. In some embodiments, the lipid shell is a lipid bilayer.

In some embodiments, the reservoir molecule is not a lipid.

Each of the limitations described herein can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The disclosure is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows the synthesis of Nitric Oxide HDL NPs (NO-HDL NPs). The top panel shows S-nitrosylation of DPPTE. The bottom panel shows the synthesis of NO HDL NPs.

FIGS. 2A-2E show the characterization of NO-HDL NPs. FIG. 2A shows a SNO-DPPTE mass spectrograph. FIGS. 2B and 2C show fold excess SNO DPPTE per AuNP vs. SNO/HDL NP. FIG. 2D shows a graph of relative absorbance arising from the NO-HDL NPs as a percentage of control. FIG. 2E shows a graph of the percent SNO remaining.

FIG. 3 shows a mouse renal transplantation model of ischemia-reperfusion injury (IRI).

FIG. 4 shows HDL NPs and NO-HDL NPs reduce ischemia-reperfusion injury in a mouse renal transplant model.

FIGS. 5A-5D show the characterization of SNO-PL. FIG. 5A shows the reaction scheme for production of SNO DPPTE. FIG. 5B shows the UV/Vis spectra for DPPTE and SNO-PL, with the S—N═O peak at 335 nm. FIG. 5C (FTIR spectra) and FIG. 5D (Raman spectra) demonstrate conversion of an —SH group of DPPTE to an —S—N═O group in SNO-PL.

FIGS. 6A-6C show in vitro stability, toxicity and efficacy of SNO HDL NPs. FIG. 6A shows that the SNO group on SNO HDL NPs was stable when stored at +4° C. for up to 50 days before appreciably decreasing. *p<0.05 v. Day 1. FIG. 6B shows the toxicity of SNO HDL NPs and HDL NPs on HAEC and AoSMCs. FIG. 6C shows that SNO HDL NPs reduce migration of AoSMCs. *p<0.05 v. PBS and SNO HDL NP; **p<0.05 v. PBS and HDL NP.

FIGS. 7A-7B show an in vivo model of kidney transplantation. FIG. 7A shows plasma creatinine levels of mouse kidney transplant recipients on day 2 post transplantation. *p<0.05 v. PBS control. FIG. 7B shows immunocytochemistry for Gr-1 (light gray), a neutrophil marker, in representative sections of PBS, HDL NP and SNO HDL NP treated kidney recipients. The dark gray stain is DAPI.

FIGS. 8A-8B show reaction kinetics and stoichiometry of S-nitrosylation of DPPTE. FIG. 8A shows how the phospholipid DPPTE and sodium nitrite were added at various ratios and the S-nitrosylation reaction was monitored using a UV/Vis spectrophotometer. FIG. 8B shows mass spectroscopy analysis of phospholipid to nitrite combinations.

FIG. 9 shows UV/Vis spectra of HDL NP and SNO HDL NP. UV/Vis spectra of HDL NP and SNO HDL NP constructs demonstrates a local maximum at ˜520 nm. The SNO peak at 335 nm in the SNO HDL NP is not visible due to background signal from the HDL NP.

FIG. 10 shows representative images of the AoSMC transwell migration assay, showing crystal violet stained AoSMC cells following transwell migration.

FIG. 11 shows TUNEL staining of rransplanted kidney grafts on Day 2. Representative images of TUNEL staining (PBS— light gray; HDL NP and SNO-HDL NP-medium gray) in transplanted kidney grafts are shown. Nuclei are counter-stained with DAPI (dark gray).

FIG. 12 shows Ki67 staining of transplanted kidney grafts on Day 2. Kidney grafts were stained for Ki67, a proliferation marker. Light gray is Ki67 and dark gray is nuclei (DAPI).

FIG. 13 shows macrophage staining of transplanted kidney grafts on Day 2. Representative images of transplanted kidney grafts stained for F4/80, a macrophage marker are shown. Light gray is F4/80 and dark gray is nuclei (DAPI).

FIG. 14 shows the S-nitrosylation of DPPTE. The final product has an absorbance peak at 335 nm.

FIG. 15 shows the absorbance (AU) of the S-nitrosylation reaction at 335 nm (left panel) and the S-nitrosylation reaction velocity (right panel).

FIG. 16 shows a mouse renal transplant model. It measures plasma creatinine as a marker of kidney ischemia and reperfusion injury.

FIG. 17 is a graph showing that HDL NP and SNO HDL NP demonstrate a decrease in plasma creatine on Day 2.

FIG. 18 shows kidney transplant histology using TUNEL (apoptosis) and Gr-1 (neutrophils) staining.

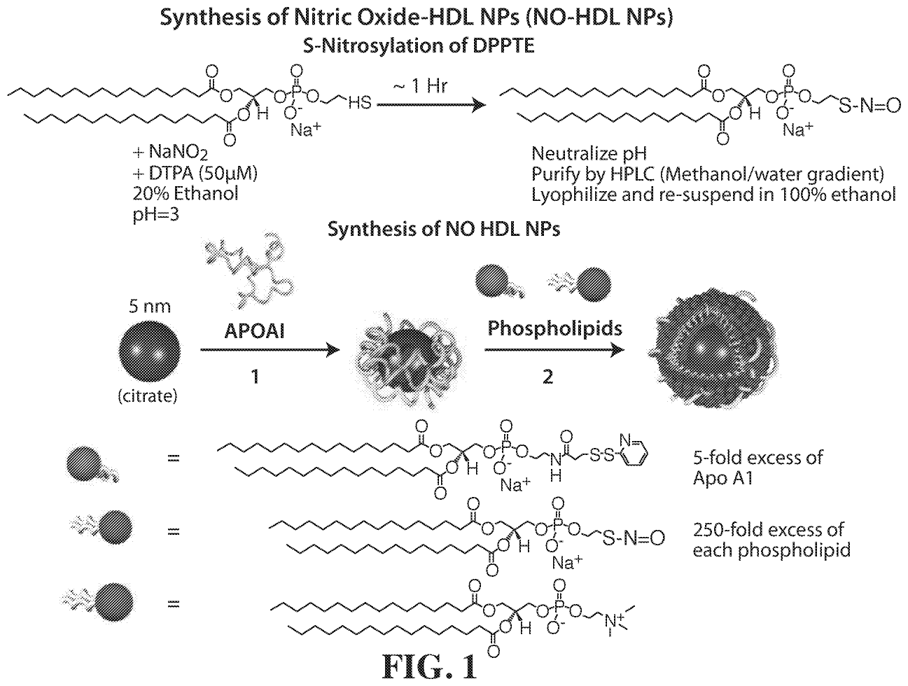

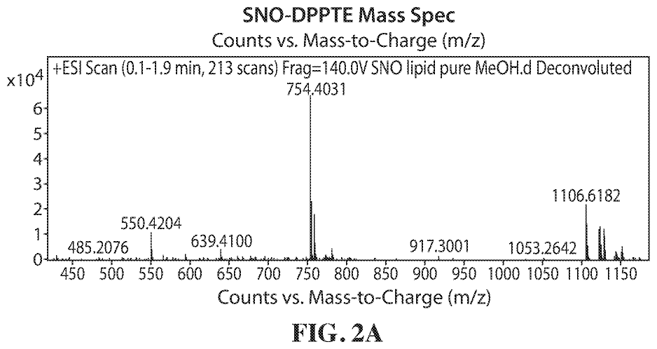

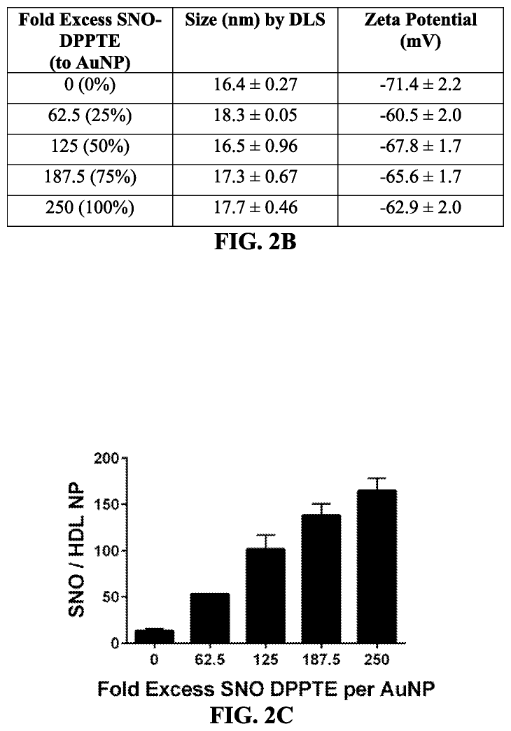

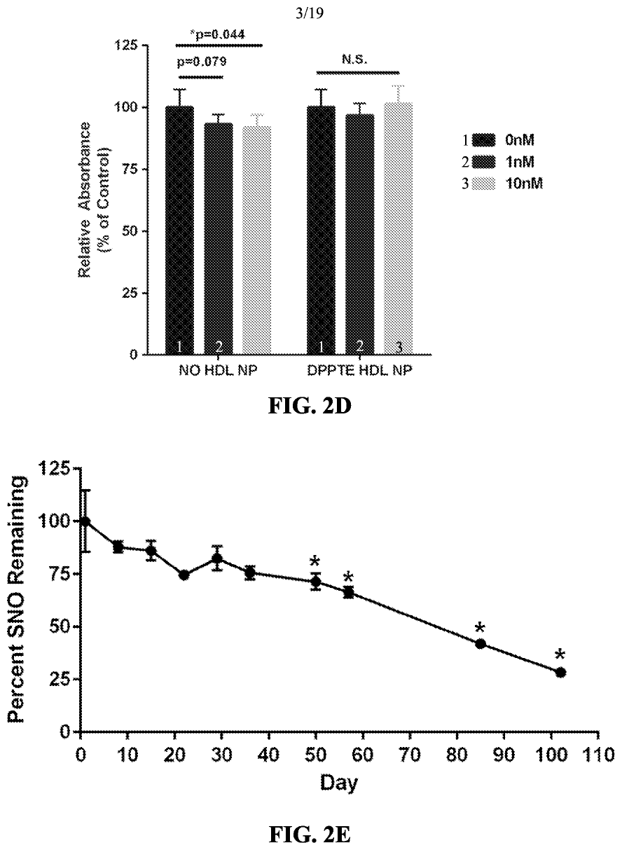

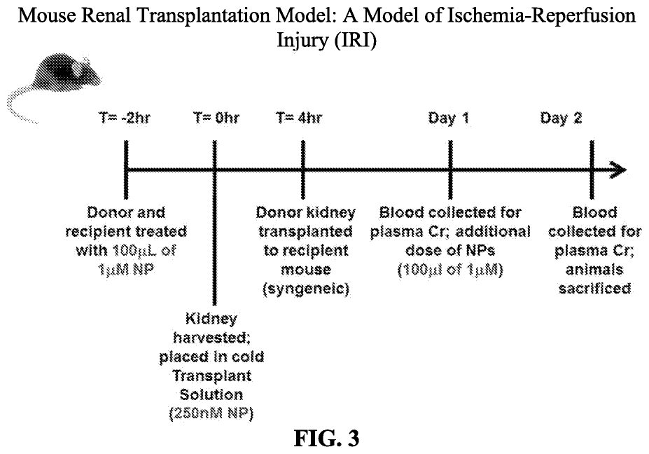

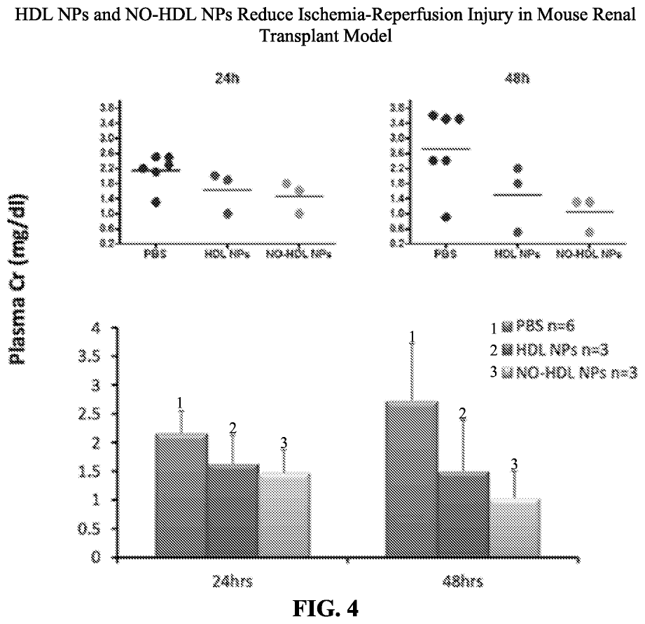

### DETAILED DESCRIPTION

The invention described herein, in some aspects, is a versatile platform for targeted delivery of NO, based on synthetic high-density lipoprotein nanoparticles (HDL-NPs). Nanostructures are synthesized using a nanoparticle core, such as a gold core, to control size and shape, and modified lipids that harbor NO and serve as NO releasing nanoparticles. NO releasing high density lipoprotein nanoparticles have been designed with similar characteristics to natural HDL (the ‘good’ cholesterol). The NPs in some aspects contain molecules such as phospholipids modified to release NO, as well as regenerate their NO group through interaction with the amino acid arginine. These materials may be used as treatment for diseases of cholesterol overload, in instances of revascularization, or as therapy in any case of where ischemia-reperfusion injury is suspected.

In aspects, the present invention generally relates to the prevention of restenosis following vascular interventions (e.g., angioplasty), the reduction of ischemia-reperfusion injury following myocardial infarction and/or organ transplantation, prolonging of cold ischemia time of donor organs, the reduction of atherosclerotic plaque burden, ameliorating endothelial dysfunction and stiffening in atherosclerosis development, and as a therapy for blood pressure.

The present invention has advantages including, but not limited to, S-nitrosylation of the phospholipid in outer leaflet of HDL NPs, which allows the nanoparticles to deliver NO to locations targeted by HDL NPs (e.g., SR-B1 expressing cells), improving biomimetic nanoparticle design, stabilizing nanoparticle formulation, and allowing a large number of phospholipids on the outer leaflet of lipid bilayer creating a large number of S-nitrosylated phospholipids per nanoparticle.

Nitric Oxide Nitric oxide (NO) is a gaseous signaling molecule with fundamental actions in biology with numerous regulatory, protective and therapeutic properties. In higher vertebrates it has key roles in maintaining homeostasis and in smooth muscle (especially vascular smooth muscle), neurons and the gastrointestinal tract. NO is involved in regulating aspects from waking, digestion, sexual function, perception of pain and pleasure, memory recall and sleeping. The way NO functions in the body influences how humans degenerate with age. NO also plays a key role in cardiovascular disease, stroke, diabetes, and cancer. Thus, the ability to control NO signaling and to use NO effectively in therapy presents a major bearing on the future quality and duration of human life.

NO is produced from L-arginine by nitric oxide synthase (NOS). The NOS of the human body has three NOS isomers. The different NOS isoforms exhibit tissue- and cell-type specific distributions and activities, which reflect their specific physiological roles. eNOS is active primarily in the endothelial tissue of blood vessels, where NO mediates vasodilation and relaxation of soft tissue (Moncada et al. (2006) J Neurochem 97: 1676-1689). eNOS is a constitutively active isoform that produces low levels of NO at a steady rate over long periods to achieve its functional roles (Moncada et al., (2006) J Neurochem 97:1676-1689). iNOS is active primarily in immune cells and glial cells and is activated by pathogen recognition and cytokine release (Moncada et al. (2006) J Neurochem 97:1676-1689; Merrill et al. (1997) J Neurosci Res 48:372-384). The primary function of iNOS is to mediate cell death in response to pathogens by generating NO at toxic levels. Thus, iNOS produces high concentrations of NO over short periods (Knott et al. (2009) Antioxid Redox Signal 11: 541-554). nNOS is active primarily in central and peripheral neurons where NO serves as an important neurotransmitter in cell-to-cell communication and neuronal plasticity (Knott et al. (2009) Antioxid Redox Signal 11:541-554) Similar to eNOS, nNOS is constitutively active and produces low levels of NO over long periods. Finally, mtNOS is the most recently identified member of the NOS family (Ghafourifar et al. (2005) Trends Pharmacol Sci 26:190-195). mtNOS localizes to the mitochondrial inner membrane and plays a role in the regulation of bioenergetics and Ca²⁺ buffering (Ghafourifar et al. (1997) FEBS Lett 418:291-296).

NO contributes to various pathologies through formation of reactive nitrogen species (RNS) and modification of proteins and also plays important physiological roles in blood vessel dilation, neurotransmission and immune cell response. NO was first identified as the endothelium-derived relaxing factor that mediates blood vessel dilation (Ignarro et al. (1987) Proc Natl Acad Sci USA 84:9265-9269). In addition, NO is involved in multiple nervous system activities including nerve-mediated relaxation of the gut during digestion (Snyder et al. (1992) Science 257:494-496), innervation of neural blood vessels in cerebral and penile arteries (Bredt et al. (1991) Neuron 7:615-624; Bredt et al. (1991) Nature 351:714-718; Burnett et al. (1992) Science 257:401-403) and prevention of excitotoxicity by S-nitrosylation of N-methyl-d-aspartate (NMDA) glutamate receptors (Choi et al. (2000) Nat Neurosci 3:15-21; Kim et al. (1999) Neuron 24:461-469).

Augmenting the body's natural generation of NO by either stimulating increased production of endogenous NO or introducing exogenously-produced NO into the body can improve the body's response to damage, pain, and invading organisms. However, it is difficult to deliver NO into living tissue. To be clinically useful, NO must be present in the site of action in a sufficient quantity.

Methods in the prior art for delivering NO for therapeutic purposes include the administration of chemical compounds which release NO chemically into the body. Other methods employ NO pathway agonists and NO antagonists. Still other methods employ high pressure NO gas and sprays. Yet another method involves surrounding a body with sealed vacuum containers into which gaseous NO is introduced. Attempts have also been made to force pressurized NO through tissue and skin. For various reasons, these methods have yielded limited results. For example, gaseous NO is highly reactive, has low diffusion constant and has extremely short life-time in tissue media.

There are several solutions that target specific clinical outcomes involving NO. Sildenafil citrate (sold under the brand name VIAGRA®), for example, interferes with the down regulation of NO in erectile dysfunction syndrome. Etanercept (sold under the brand name ENBRIL), for example, uses an anti-TNF alpha antibody to do what NO would do in inflammatory diseases of the joint. Most solutions involve affecting the NO pathways, due to the difficulty in stimulating production of NO directly at the site of action. Because of the lack of site specificity of these NO pathway pharmacologics, negative side effects can be detrimental.

NO plays an active defense role in the immune system. It is a strong antioxidant, and can suppress bacterial infections, viruses and parasitic attacks. NO can be used to reduce inflammation, facilitate vasodilation, alleviate pain associated with joint swelling in arthritis, including but not limited to, pain associated with osteoarthritis and Rheumatoid Arthritis, combating Gram Positive microorganisms, Gram Negative microorganisms, Fungi (including onychomycosis) and viruses. It is also therapeutic in treating osteoporosis, collagen formation, stem cell signaling, satellite cell differentiation, wound-healing, wound-management, reduction in scar tissue, remediation of activity related injury, and acne. It can even deter some types of cancer cell growth and inhibit cancer cell proliferation. NO can also enhance nerve regeneration, promote apoptosis, stimulate endogenous NO production, and stimulate iNOS pathways.

NO can effectively function to maintain homeostasis in the cardiovascular and respiratory systems. NO, as a signaling molecule, causes vasodilation which promotes blood vessel flexibility, eases blood pressure, cleans the blood, reverses atherosclerosis and effectively prevents cardiovascular diseases and aids in its recovery. NO slows down atherosclerotic plaque deposition on vascular walls. In patients with moderate to severe diabetes, NO can prevent many common and serious complications. NO can effectively decrease the risk of cancer, diabetes, myocardial infarction and stroke. In the respiratory system, NO dilates blood vessels in the lungs, improving oxygenation of the blood and reducing pulmonary hypertension. Because of this, NO is provided as a therapeutic gas for patients with pulmonary hypertension.

NO can also slow the aging process and improve memory. The NO molecules produced by the immune system are not only capable of destroying invading microorganisms, but also help activate and nourish brain cells, significantly slowing aging and improving memory.

Besides s-nitrosylation (e.g., nitrosylated lipid), another non-limiting example of a modification to generate a NO-donating group is nitrosylation of a nitrogen (N-nitrosylation) to provide an N-nitrosylated molecule (e.g., a lipid). In some embodiments, the reservoir molecule is a lipid molecule that has been modified to include other molecules that can donate an NO group. Non-limiting examples of other molecules include diazeniumdiolates (also known as NONOates) (See e.g., Ramamurthi et al. (1997) Chem Res Toxicol 10(4):408-413). Diazeniumdiolates typically have half-lives of milliseconds in biological systems (e.g., cell culture media, plasma, etc.). The reservoir molecule (e.g., nitrosylated lipid) is able to release a NO group at a target site. In some embodiments, the reservoir molecule is not a lipid. Non-limiting examples of non-lipid reservoir molecules, include but are not limited to, glutathione (See e.g., Pompella et al., Biochem Pharmacol 2003 66(8):1499-1503). Glutathione is a tripeptide that acts as a natural NO reservoir in vivo. In some embodiments, the structure, nanostructure or nanoparticle (e.g., HDL nanoparticle) described herein contains one or more glutathiones. In some embodiments, the free thiol in glutathione is modified (e.g., S-nitrosylated).

Other non-limiting examples of NO donors include L-arginine and L-arginine hydrochloride, D,L-arginine, D-arginine, or alkyl (e.g., ethyl, methyl, propyl, isopropyl, butyl, isobutyl, tert-butyl, etc.) esters of L-arginine and/or D-arginine (e.g., a methyl ester, an ethyl ester, a propyl ester, a butyl ester, etc.) and/or salts thereof, as well as other derivatives of arginine and other NO donors. For instance, non-limiting examples of pharmaceutically acceptable salts include hydrochloride, glutamate, butyrate, or glycolate (e.g., resulting in L-arginine glutamate, L-arginine butyrate, L-arginine glycolate, D-arginine hydrochloride, D-arginine glutamate, etc.). Other examples of NO donors include L-arginine-based compounds such as, but not limited to, L-homoarginine, N-hydroxy-L-arginine, nitrosylated L-arginine, nitrosylated L-arginine, nitrosylated N-hydroxy-L-arginine, nitrosylated N-hydroxy-L-arginine, citrulline, omithine, linsidomine, nipride, glutamine, etc., and salts thereof (e.g., hydrochloride, glutamate, butyrate, glycolate, etc.). Still other non-limiting examples of NO donors include S-nitrosothiols, nitrites, 2-hydroxy-2-nitrosohydrazines, or substrates of various forms of NOS. In some cases, the NO may be a compound that stimulates endogenous production of NO in vivo. Examples of such compounds include, but are not limited to, L-arginine, substrates of various forms of NOS, certain cytokines, adenosine, bradykinin, calreticulin, bisacodyl, phenolphthalein, OH-arginine, or endothelein. It should be understood that, in any of the embodiments described herein that describe a S-nitrosylated lipid, other NO donors may also be used instead, or in combination with, S-nitrosylated lipids, in other embodiments of the invention.

NO plays a pivotal role in regulating vessel wall homeostasis and as such it is an important component of the vascular system.

The vascular system is made up of the vessels that carry blood and lymph through the body. The arteries and veins carry blood throughout the body, delivering oxygen and nutrients to the body tissues and taking away tissue waste matter. The lymph vessels carry lymphatic fluid. The lymphatic system helps to protect and maintain the fluid environment of the body by filtering and draining lymph away from each region of the body. The vessels of the blood circulatory system are:
(1) Arteries. Blood vessels that carry oxygenated blood away from the heart to the body.
(2) Veins. Blood vessels that carry blood from the body back into the heart.
(3) Capillaries. Tiny blood vessels between arteries and veins that distribute oxygen-rich blood to the body.

Blood moves through the circulatory system as a result of being pumped out by the heart. Blood leaving the heart through the arteries is saturated with oxygen. The arteries break down into smaller and smaller branches in order to bring oxygen and other nutrients to the cells of the body's tissues and organs. As blood moves through the capillaries, the oxygen and other nutrients move out into the cells, and waste matter from the cells moves into the capillaries. As the blood leaves the capillaries, it moves through the veins, which become larger and larger to carry the blood back to the heart.

In addition to circulating blood and lymph throughout the body, the vascular system functions as an important component of other body systems. Examples include:
(1) Respiratory system. As blood flows through the capillaries in the lungs, carbon dioxide is given up and oxygen is picked up. The carbon dioxide is expelled from the body through the lungs, and the oxygen is taken to the body tissues by the blood.
(2) Digestive system. As food is digested, blood flows through the intestinal capillaries and picks up nutrients, such as glucose (sugar), vitamins, and minerals. These nutrients are delivered to the body tissues by the blood.
(3) Kidneys and urinary system. Waste materials from the body tissues are filtered out from the blood as it flows through the kidneys. The waste material then leaves the body in the form of urine.
(4) Temperature control. Regulation of the body's temperature is assisted by the flow of blood among the different parts of the body. Heat is produced by the body's tissues as they go through the processes of breaking down nutrients for energy, making new tissue, and giving up waste matter.

A vascular disease is a condition that affects the arteries and/or veins. Most often, vascular disease affects blood flow, either by blocking or weakening blood vessels, or by damaging the valves that are found in veins. Organs and other body structures may be damaged by vascular disease as a result of decreased or completely blocked blood flow.

Causes of vascular disease include, but are not limited to:
(1) Atherosclerosis. Atherosclerosis (a buildup of plaque, which is a deposit of fatty substances, cholesterol, cellular waste products, calcium, and fibrin in the inner lining of an artery) is the most common cause of vascular disease. It is unknown exactly how atherosclerosis begins or what causes it. Atherosclerosis is a slow, progressive, vascular disease that may start as early as childhood. However, the disease has the potential to progress rapidly. It is generally characterized by the accumulation of fatty deposits along the innermost layer of the arteries. If the disease process progresses, plaque formation may take place. This thickening narrows the arteries and can decrease blood flow or completely block the flow of blood to organs and other body tissues and structures.
(2) Embolus/thrombus. A blood vessel may be blocked by an embolus (a tiny mass of debris that moves through the bloodstream) or a thrombus (a blood clot).
(3) Inflammation. In general, inflammation of blood vessels is referred to as vasculitis, which includes a range of disorders. Inflammation may lead to narrowing and/or blockage of blood vessels.
(4) Trauma/injury. Trauma or injury involving the blood vessels may lead to inflammation or infection, which can damage the blood vessels and lead to narrowing and/or blockage.

Because the functions of the blood vessels include supplying all organs and tissues of the body with oxygen and nutrients, removal of waste products, fluid balance, and other functions, conditions that affect the vascular system may affect the part(s) of the body supplied by a particular vascular network, such as the coronary arteries of the heart.

As a free radical gas, NO has a short half-life. In certain instances, it may be desirable to increase the effective amount of NO in a cell, tissue, or organ in order to induce vascular relaxation, vascular dilation, vascularization, oxygenation, or other NO mediated biological process. The compositions and formulations of the present invention may be used in combination with either conventional methods of treatment or therapy or may be used separately from conventional methods of treatment or therapy. When the compositions and formulations of the present invention are administered in combination therapies with other agents, they may be administered sequentially or concurrently to an individual. Alternatively, pharmaceutical compositions according to the present invention include a combination of a NO releasing HDL-NP of the present invention optionally in association with a pharmaceutically acceptable excipient, as described herein, and another therapeutic or prophylactic agent known in the art.

NO Deficiency Disorders

The compositions of the invention are useful in treating disorders resulting from NO deficiency or disorders that cause NO deficiency. Reasons for NO deficiency include but are not limited to: 1) NOS dysfunction, resulting in the inability to produce NO from L-arginine in the blood vessels; 2) poor diet with insufficient nitrates and/or excess sugar intake; 3) oral dysbiosis or the inability of oral bacteria to convert dietary sources of nitrate into NO; 4) genetic disorder or weakness that affect NO production (e.g., endothelial dysfunction, argininosuccinic aciduria, Huntington's disease, sickle cell disease, hyperhomocystinemia, acute chest syndrome, muscular dystrophy, dyslipidemia, hypertensive disorders of pregnancy (e.g., pre-eclampsia), or senescence (e.g., Alzheimer's disease)); and 5) sedentary lifestyle.

The compositions of the invention are useful in improving learning and memory related to aging and protecting the skin from sun damage. NO deficiency plays a definite role in aging. Aging can cause >50% loss in endothelial function. Further, a loss of 75% of endothelium derived NO is seen in 70-80 year old subjects compared to a younger population of subjects. Abnormal vasodilation in certain arteries also occurs with aging. Collectively, these findings illustrate that endothelial function declines progressively with age, as a consequence of declining NO levels in healthy subjects as well as subjects with existing diseases or disorders. Reduced availability of NO may increase risk of cardiovascular disease, sexual dysfunction and Alzheimer's Disease. Aging impairs the mechanism through which NO in the brain induces sleep. Reduced NO production and impaired endothelia function is observed in obstructive sleep apnea (OSA).

The compositions of the present invention are also useful in relieving the symptoms of NO deficiency. Many symptoms of NO insufficiency occur with age: loss of energy, loss of memory, decline in sexual health and performance, and aches and pains that over time can manifest as specific disease.

In some embodiments, a subject may be diagnosed with, or otherwise known to have, a disease or bodily condition associated with a NO mediated disorder. A NO mediated disorder is any disorder that is affected with NO therapy. NO mediated disorders include but are not limited to vascular conditions, diseases or disorders, as described herein. Vascular conditions, diseases or disorders include, but are not limited to, neurological disease, autoimmune disease, diseases of inflammation, diseases of blood vessels, angiogenesis, atherosclerosis, high blood pressure, kidney disease, cancer, cardiovascular disease, peripheral vascular disease, disease of the central nervous system, degenerative diseases, rheumatic diseases, connective tissue diseases, ischemia, tissue reperfusion, transplantation, infectious disease, thrombosis, diseases of blood clotting, hypercoagulation, platelet disorders, neutrophil disorders, disorders of white blood cells, endothelial disease, heart disease, erectile dysfunction, disorders of low blood flow and/or pulmonary disease. In some embodiments, the subject may be diagnosed with diseases related to cholesterol overload, revascularization, and/or in any case of where ischemia-reperfusion injury is suspected. In some embodiments, the subject may be diagnosed with, or otherwise known to have, a disease or bodily condition related to vascular injury, atherosclerosis, restenosis following vascular interventions (e.g. angioplasty), ischemia/reperfusion injury, ischemia-reperfusion injury following myocardial infarction and/or organ transplantation, prolong cold ischemia time of donor organs, atherosclerotic plaque burden, endothelial dysfunction and stiffening in atherosclerosis development, and/or disorders of blood pressure. In some embodiments, the subject may be diagnosed with, or otherwise known to have, a disease or bodily condition treated by precutaneous balloon angioplasty, stent placement, or disorders of blood vessel remodeling after procedures such as neointimal hyperplasia.

Cardiovascular Disease

The compositions of the present invention may be used to treat cardiovascular disease. Cardiovascular disease is a vascular endothelial cell dysfunction and certain symptoms begin, including as conventional or above the heart and vascular system-on, atherosclerosis, hypertension, gojihyeol, coronary heart disease (heart attack), cerebrovascular diseases (stroke, dementia), peripheral vascular disease, arrhythmia, heart failure, congestive heart disease Chung, cardiac disease and for at least the name of the heart and blood vessels, including, but not limited thereto.

As the main factors of cardiovascular disease expression of genetic factors, lifestyle habits, such as known very diverse complications of diabetes, but the endothelial cell type NOS reduction of NO and the active oxygen species (ROS) are known to increase due to the increase in vascular oxidative stress. Endothelial cell-type NO produced by the NOS is a powerful vasodilator factors, while platelet aggregation, vascular muscle cell proliferation, the mononuclear cell vascular deposition, by inhibiting the atherosclerosis-related protein so the homeostasis of the whole cardiovascular system play an important role (Forstermann et al. (2006) Circulation 113:1708-1714). However, due to the generation of ROS within the blood vessel due to a number of factors to increased activity of the various enzymes responsible for the generation of NOx is reduced (Gryglewski et al. (1986) Nature 320:454-456; Paravicini et al. (2002) Circulation Research 91:54-61; Dusting et al. (1998) Clinical and Experimental Pharmacology and Physiology 25:S34-41). In addition, production of ROS of increased vascular NO (from the damaged vascular endothelial cells of patients with clinical risk factors and coronary heart disease in atherosclerotic NO) functions associated, underlying in the blood vessel causing a contraction (Guzik et al. (2000) Cir Res 86:E85-90).

Endothelial cell dysfunction (endothelial dysfunction) was found as abnormal relaxation of the blood vessels in patients with hypertension in 1990 (Panza J A et al. (1990) New England Journal of Medicine 323:22-27). High blood pressure, arteriosclerosis, hyperlipidemia, diabetes, obesity are comprehensive primary function disorders that further add to cardiovascular disease. (Brunner et al. (2005) J. Hypertens 23:233-246). As epithelial cells, endothelial cells that line along the heart, blood vessels and the lymphatic cavitiesproduce a vasodilator and vasoconstrictor nerve agents to adjust both the vascular tone and structure. NO carries a variety of functions in the maintenance of vascular homeostasis, including the control of vascular tone, inhibition of thrombosis, inhibition of platelet aggregation, regulation of the expression of endothelial adhesion molecules.

The compositions of the invention are also useful in treating cardiovascular diseases. As used herein cardiovascular diseases included, but are not limited to, arteriosclerosis, coronary heart disease, ischemia, endothelium dysfunction, in particular those dysfunctions affecting blood vessel elasticity, restenosis, thrombosis, angina, high blood pressure, cardiomyopathy, hypertensive heart disease, heart failure, cor pulmonale, cardiac dysrhythmias, endocarditis, inflammatory cardiomegaly, myocarditis, myocardial infarction, valvular heart disease, stroke and cerebrovascular disease, aortic valve stenosis, congestive heart failure, and peripheral arterial disease. In one aspect, the invention includes methods of administering the highly bioavailable zerovalent-sulfur-rich compositions for chronic treatment. In another aspect, the invention also includes methods of administering the highly bioavailable zerovalent-sulfur-rich compositions for acute treatment.

In some embodiments, the compositions of the invention will restore and/or improve cardiovascular parameters to normal ranges in a subject diagnosed with or at risk of a cardiovascular disease. Normal ranges of cardiovascular parameters include but are not limited to, an end-diastolic volume (EDV) from about 65-240 mL, an end-systolic volume (ESV) from about 16-143 mL, a stroke volume from about 55-100 mL, an ejection fraction from about 55-70%, a heart rate from about 60-100 bpm, and/or cardiac output of about 4.0-8.0 L/min. NO HDL NPs would improve patient survival and outcomes following vascular interventions (e.g. angioplasty) as well as possibly preventing myocardial infarction-induced heart damage.

Inflammatory Disease

The compositions of the invention may also be used to treat inflammatory diseases. Examples of inflammatory diseases include, but are not limited to acne vulgaris, asthma, autoimmune diseases (e.g., acute disseminated encephalomyelitis (ADEM), Addison's disease, agammaglbulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmunehemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo concentric sclerosis, Behcet's disease, Berger's disease, Bickerstaff's encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's syndrome, cutaneous leukocytoclastic vasculitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressive, fibrosing alveolitis, gastritis, gastrointestinal pemphigoid, giant cell arteritis, glomerulonephritis, Goodpasture's syndrome, Grave's disease, Guillain-Barre syndrome, Hashimoto's encephalopathy, Hashimoto's thyroiditis, Henoch-Schonlein purpura, herpes gestationis, hidradenitis suppurativa, Hughes-Stovin syndrome, hypogammaglobulinemia, idiopathic inflammatory demyelinating diseases, idiopathic pulmonary fibrosis, idiopathic thrombocytopenic purpura, IgA nephropathy, inclusion body myositis, chronic inflammatory demyelinating polyneuropathy, interstitial cystitis, juvenile idiopathic arthritis, Kawasaki's disease, Lambert-Eaton myasthenic syndrome, leukocytoclastic vasculitis, lichen planus, lichen sclerosus, linear IgA disease, lupus erythematosus, Majeed syndrome, Meniere's disease, microscopic polyangiitis, mixed connective tissue disease, morphea, Mucha-Habermann disease, myasthenia gravis, myositis, narcolepsy, neuromyelitis optica, neuromyotonia, ocular cicatricial pemphigoid, opsoclonus myoclonus syndrome, Ord's thyroiditis, palindromic rheumatism, PANDAS, paraneoplastic cerebellar degeneration, paroxysmal nocturnal hemoglobinuria, Parry Romberg syndrome, Parsonage-Turner syndrome, pars planitis, pemphigus vulgaris, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriatic arthritis, pyoderma gangrenosum, pure red cell aplasia, Rasmussen's encephalitis, raynaud phenomenon, relapsing polychondritis, Reiter's syndrome, restless leg syndrome, retroperitoneal fibrosis, rheumatic fever, Schnitzler syndrome, scleritis, scleroderma, serum sickness, Sjogren's syndrome, spondyloarthropathy, stiff person syndrome, subacute bacterial endocarditis, Susac's syndrome, Sweet's syndrome, sympathetic ophthalmia, Takayasu's arteritis, temporal arteritis, thrombocytopenia, Tolosa-Hunt syndrome, transverse myelitis, ulcerative colitis, undifferentiated connective tissue disease, undifferentiated spondyloarthropathy, vitiligo, and Wegener's granulomatosis), celiac disease, chronic prostatitis, glomerulonephritis, hypersensitivities, inflammatory bowel diseases, pelvic inflammatory disease, reperfusion injury (including, but not limited to ischemia reperfusion injury following organ transplantation), rheumatoid arthritis, sarcoidosis, transplant rejection, vasculitis, interstitial cystitis, and osteoarthritis and other pathological conditions associated with oxidative stress and/or an imbalance in redox homeostasis.

The compositions of the invention may be useful in treating other conditions associated with oxidative stress including but not limited to autism, schizophrenia, bipolar disorder, fragile X syndrome, sickle cell disease, chronic fatigue syndrome, osteoarthritis cataract, macular degeneration, toxic hepatitis, viral hepatitis, cirrhosis, chronic hepatitis, oxidative stress from dialysis, renal toxicity, kidney failure, ulcerative colitis, bacterial infection, viral infections, such as HIV and AIDS, herpes, ear infection, upper respiratory tract diseases, hypertension, balding and hair loss, over-training syndrome related to athletic performance, eczema, scleroderma, atopic dermatitis, polymyositis, and dermatitis herpetiformis.

Diabetes

The compositions of the invention may also be useful for treating diabetes and its complications. Diabetes can be any metabolic disease in which a person has high blood sugar, either because the body does not produce enough insulin, or because cells do not respond to the insulin that is produced. Non-limiting examples of diabetes includes, type 1 diabetes mellitus, type 2 diabetes mellitus, gestational diabetes, congenital diabetes, cystic fibrosis-related diabetes, steroid diabetes, latent autoimmune diabetes of adults, and monogenic diabetes. Complications associated with diabetes include but are not limited to hypoglycemia, diabetic ketoacidosis, nonketotic hyperosmolar coma, cardiovascular disease, chronic renal failure, diabetic nephropathy, diabetic neuropathy, diabetes-related foot problems (e.g., diabetic foot ulcers), and diabetic retinopathy.

Cancer

Other conditions that may be treated using compositions of the invention include cancers. Cancers are generally characterized by unregulated cell growth, formation of malignant tumors, and invasion to nearby parts of the body. Cancers may also spread to more distant parts of the body through the lymphatic system or bloodstream. Cancers may be a result of gene damage due to tobacco use, certain infections, radiation, lack of physical activity, obesity, and/or environmental pollutants. Cancers may also be a result of existing genetic faults within cells to cause diseases due to genetic heredity. Screenings may be used to detect cancers before any noticeable symptoms appear and treatment may be given to those who are at higher risks of developing cancers (e.g., people with a family history of cancers). Examples of screening techniques for cancer include but are not limited to physical examination, blood or urine tests, medical imaging, and/or genetic testing. Non-limiting examples of cancers include: bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer.

Organ Transplantation

The compositions of the present invention may be useful to treat graft (e.g., organ, tissue, etc.) rejection. An organ transplant surgery replaces a failing organ with a healthy organ. The success rates of transplant surgery has improved from its start, but growing shortages exist in the supply of organs and tissues available for transplantation. Transplants may be the patient's own tissue (autografts; e.g., bone, bone marrow, and skin grafts); genetically identical (syngeneic or between monozygotic twins) donor tissue (isografts); genetically dissimilar donor tissue (allografts, or homografts); or, rarely, grafts from a different species (xenografts, or heterografts). Transplanted tissue may be cells (e.g., hematopoietic stem cell [HSC], lymphocyte, and pancreatic islet cell transplants, etc.); parts or segments of an organ (e.g., hepatic or pulmonary lobar transplants and skin grafts, etc.), entire organs (e.g., heart, lung, kidney, liver, pancreas, intestine, stomach, testis, hand transplants, etc.), tissues (e.g., cornea, skin, islets of Langerhans, bone marrow, blood, blood vessels, heart valve, bone, composite tissue grafts, etc.). Tissues may be grafted to an anatomically normal site (orthotopic; e.g., heart transplants) or abnormal site (heterotopic; e.g., a kidney transplanted into the iliac fossa). With rare exceptions, clinical transplantation uses allografts from living related, living unrelated, or deceased donors. Living donors are often used for kidney and HSC transplants and less frequently for segmental liver, pancreas, and lung transplants. Use of deceased-donor organs (from heart-beating or non-heart-beating donors) has helped reduce the disparity between organ demand and supply; however, demand still far exceeds supply, and the number of patients waiting for organ transplants continues to grow.

Organ and tissue transplantation is the preferred clinical approach to treat patients suffering from organ failure or complications arising from diseases of specific organs and tissues. However, transplant patients face a lifetime of immunosuppressive therapy and the risk of losing the new organ due to rejection. Although improvements have been made in the transplantation process, rejection remains the most common complication following transplantation and is the major source of morbidity and mortality. Transplant rejection occurs when the immune system of the recipient of a transplant attacks the transplanted organ or tissue. Rejection is an adaptive immune response and is mediated through both T lymphocyte-mediated and humoral immune (antibodies) mechanisms.

Donor organs are mostly stored in a cold environment for preservation (e.g., static cold preservation) because the metabolic rate of eukaryotic cells decline from two to three times at 10° C. of reduction in the temperature in which they are. The technique requires the blood fast removal, fast organ cooling and a balance between the preservation solution and the organ. The preservation conditions are stressful and may cause damages resulting from ischemia (preservation hypothermic conditions) and reperfusion (transplantation in the donor). The preservation technique in hypothermic conditions has been applied first in 1952 by Lefevbre and Nizet, in France. Since then, only a few advances have been achieved in the organs preservation.

All allograft recipients are at risk of graft rejection; the recipient's immune system recognizes the graft as foreign and seeks to destroy it. Rejection of solid organs may be hyperacute, accelerated, acute, or chronic (late). These categories can be distinguished histopathologically and approximately by the time of onset. Symptoms vary by organ. Recipients of grafts containing immune cells (particularly e.g., bone marrow, intestine, and liver) are at risk of graft-vs-host disease (GVHD). GVHD occurs when donor T cells react against recipient's self-antigens. It can include inflammatory damage to tissues, especially the liver, intestine, and skin, as well as blood dyscrasia (Information available from www.merckmanuals.com/professional/immunology-allergic-disorders/transplantation/overview-of-transplantation). Organ rejection and/or GVHD may occur after heart, heart valve, lung, kidney, liver, pancreas, intestine, skin blood vessel, bone marrow, stem cell, bone, or islet cell transplantation. An islet cell transplantation can be performed to prevent the onset of diabetes or as a treatment of diabetes (Information available from U.S. Application Publication No. 2016/0311914).

Current methods to reduce the risk of these complications is minimized by pre-transplantation screening and immunosuppressive therapy during and after transplantation. The immunotherapy for solid organ transplantation is primarily T lymphocyte-directed and focused on preventing acute rejection. Immunosuppressants are primarily responsible for the success of transplantation. Treatment regimens include corticosteroids, calcineurin inhibitors (CNIs; e.g., cyclosporine, tacrolimus), cyclosporine, tacrolimusis, purine metabolism inhibitors (e.g., azathioprine and mycophenolate mofetil), rapamycins (e.g., sirolimus, everolimus), immunosuppressive immunoglobulins (e.g, antilymphocyte globulin [ALG], antithymocyte globulin [ATG]), monoclonal antibodies (mAbs; e.g., mAbs directed against T cells, OKT3, anti-IL-2 receptor monoclonal antibodies), irradiation. However, immunosuppressants suppress all immune responses and contribute to many posttransplantation complications, including development of cancer, acceleration of cardiovascular disease, and even death due to overwhelming infection. Allograft survival rates in the non-sensitized, cross-match negative recipient are quite good. However, long-term allograft survival rates remain unsatisfactory; which demonstrates that transplantation tolerance remains an unfulfilled goal. Thus, there remains a need for methods to promote organ or tissue transplantation tolerance in patients.

The immunosuppressive drugs currently used for the therapeutic treatment and handling of the organs rejection are focused on the inhibition of the alloreactive cell activation. However, they have several problems related to the induction of severe side effects. Among the severe side effects are hypertension, nephrotoxicity, central nervous system dysfunction (e.g., shivering, headache, depression, paresthesia, blurry vision), increased risk of viral, bacterial or fungal infections, increased risk of tumors occurrence, lack of appetite, nausea; some patients are resistant to the drugs and the combination of several drugs is necessary; high cost of the drugs; some drugs demonstrate adverse interactions with other drugs, such as antibiotics, non-steroidal anti-inflammatory, antiepileptic, antifungal and also immunization, such as German measles and polio.

Kidney transplantation is the most common type of solid organ transplantation. More than one half of donated kidneys come from previously healthy, brain-dead individuals. About one third of these kidneys are marginal, with physiologic or procedure-related damage, but are used because demand is so great. More kidneys from non-heart-beating donors (called donation-after-cardiac-death [DCD] grafts) are being used. These kidneys may have been damaged by ischemia before the donor's death, and their function is often impaired because of acute tubular necrosis; however, over the long term, they seem to function as well as kidneys from donors that meet standard criteria (called standard criteria donors [SCD]). The remaining donated kidneys (about another 40%) come from living donors; because of limited supply, allografts from carefully selected living unrelated donors are being increasingly used. Living donors relinquish reserve renal capacity, may put themselves at risk of procedural and long-term morbidity, and may have psychologic conflicts about donation; therefore, they are evaluated for normal bilateral renal function, absence of systemic disease, histocompatibility, emotional stability, and ability to give informed consent. Use of kidneys from unrelated living donors has been increasing; kidney exchange programs often match a prospective donor and recipient who are incompatible with other similar incompatible pairs. When many such pairs are identified, chain exchanges are possible, greatly increasing the potential for a good match between recipient and donor.

The donor kidney is removed during a laparoscopic (or rarely, an open) procedure, perfused with cooling solutions containing relatively large concentrations of poorly permeating substances (eg, mannitol, hetastarch) and electrolyte concentrations approximating intracellular levels, then stored in an iced solution. Kidneys preserved this way usually function well if transplanted within 24 h. Although not commonly used, continuous pulsatile hypothermic perfusion with an oxygenated, plasma-based perfusate can extend ex vivo viability up to 48 h.

Immunosuppressive regimens vary. Commonly, calcineurin inhibitors are begun immediately after transplantation in doses titrated to minimize toxicity and rejection while maintaining trough blood levels high enough to prevent rejection. On the day of transplantation, IV or oral corticosteroids are also given; dose is tapered over the following weeks depending on the protocol used. Despite use of immunosuppressants, about 20% of kidney transplant recipients have one or more rejection episodes within the first year after transplantation. Most episodes are easily treated with a corticosteroid bolus; however, they contribute to long-term insufficiency, graft failure, or both. Signs of rejection vary by type of rejection. Chronic allograft nephropathy refers to graft insufficiency or failure ≥3 mo after transplantation. Most rejection episodes and other complications occur within 3 to 4 mo after transplantation; most patients then return to more normal health and activity but must take maintenance doses of immunosuppressants indefinitely.

At 1 yr after kidney transplantation, survival rates are in living-donor grafts: 98% (patients) and 94% (grafts); deceased-donor grafts: 95% (patients) and 88% (grafts); subsequent annual graft loss rates are 3 to 5% with a living-donor graft and 5 to 8% with a deceased-donor graft. Among patients whose graft survives the first year, half die of other causes with the graft functioning normally; half develop chronic allograft nephropathy with the graft malfunctioning in 1 to 5 yr.

In a specific patient, the most recently obtained creatinine levels should be compared with previous levels; a sudden increase in creatinine indicates the need to consider rejection or another problem (e.g., vascular compromise, obstruction of the ureter). Ideally, serum creatinine should be normal in all posttransplant patients 4 to 6 wk after kidney transplantation (Information available from the Merck Manual: www.merckmanuals.com/professional/immunology-allergic-disorders/transplantation/kidney-transplantation).

Therefore, there is a great need for novel therapies or interventions to treat organ or graft transplant rejection.

The invention, in some embodiments, provides nanostructures that deliver NO to a cell to prevent or decrease the rejection of transplanted organs. In some embodiments, the structures, nanostructures or nanoparticles described herein decrease migration of inflammatory cells (e.g., neutrophils) into the donor organ. According to some aspects, the nanostructure reduces the risk of rejection of the donor organ relative to the risk of a donor organ transplanted without exposure to the nanostructure.

Reservoir Molecule

As described herein, a “reservoir molecule” refers to a molecule with the ability to complex with NO. For instance, the reservoir molecule may be a lipid having an NO donating group. The reservoir molecule (e.g., nitrosylated lipid) is able to release a NO group at a target site. In some embodiments, the reservoir molecule is a lipid molecule that has been modified to contain a NO-donating group. A non-limiting example of a modified lipid is an S-nitrosylated lipid or N-nitrosylated lipid. In some embodiments, the reservoir molecule is a lipid molecule that has been modified to include other molecules that can donate an NO group. Non-limiting examples include diazeniumdiolates (also known as NONOates) (See e.g., Ramamurthi et al. (1997) Chem Res Toxicol 10(4):408-413). Diazeniumdiolates typically have half-lives of milliseconds in biological systems (e.g., cell culture media, plasma, etc.). The reservoir molecule (e.g., nitrosylated lipid) is able to release a NO group at a target site. In other embodiments, the reservoir molecules (e.g., heads of phospholipids) can be modified to include a wide range of moieties, including but not limited to fluorophores, MR contrast agents, be biotinylated or be glycosylated.

In other embodiments, the reservoir molecule is not a lipid. A non-limiting example of non-lipid reservoir molecules, includes but is not limited to, glutathione (See e.g., Pompella et al., Biochem Pharmacol 2003 66(8):1499-1503). Glutathione is a tripeptide that acts as a natural NO reservoir in vivo. In some embodiments, the structure, nanostructure or nanoparticle (e.g., HDL nanoparticle) described herein contains one or more glutathiones. In some embodiments, the free thiol in glutathione is modified (e.g., S-nitrosylated).

High Density Lipoprotein Nanoparticles (HDL NPs)

HDL NPs mimic natural spherical HDLs in their shape, size, surface composition (apolipoprotein A1, phospholipids), and ability to functionally efflux cholesterol from cells. Modification of the outer phospholipid, through S-nitrosylation, transforms the lipids into NO reservoirs. In addition, after release of NO, the sulfur radical can react with arginine to regenerate the S—N═O group, thus potentially allowing for sustained NO release over time.

HDL are naturally-occurring nanoparticles that assemble dynamically in serum from phospholipids, apolipoproteins, and cholesterol. HDL is involved in reverse-cholesterol transport, and has been epidemiologically correlated with reduced incidences of cardiovascular disease (Asztalos et. al.. (2011) Current Opinion in Lipidology 22:176-185; Barter et al. (2007) N Engl J Med 357:1301-1310). Natural HDL is known to bind Scavenger Receptor type B-1 (SR-B1); SR-B1 mediates uptake of cholesteryl esters and the uptake and efflux free cholesterol. Without wishing to be bound by theory, the nanoparticles, nanostructures or structures described herein may act via a specific receptor-mediated pathway, such as the SR-B1 receptor. The HDL nanoparticle is a biomimic of HDL and, as such the structures, nanostructures or nanoparticles have inherent targeting specificity to cells expressing the SR-B1 receptor. This targeting specificity for the SR-B1 receptor is conferred by both the size of the nanostructure and the presence of the ApoAl protein—a ligand for SR-B1—on the surface of the nanostructure. The nanoparticles, nanostructures or structures may also act on other receptors and/or cells.

Shell

In some aspects the invention is a structures, nanostructures or nanoparticles (e.g., HDL nanoparticles) composed of a nanostructure core of an inorganic material surrounded by a shell of a lipid layer (e.g., lipid shell), and a therapeutic agent associated with the shell. The nanostructure may also include a protein such as an apolipoprotein.

The shell may have an inner surface and an outer surface, such that the therapeutic agent and/or the apolipoprotein may be adsorbed on the outer shell and/or incorporated between the inner surface and outer surface of the shell.

The shell may also have a therapeutic profile for a therapeutic agent. A “therapeutic profile” as used herein refers to a composition of lipids and/or proteins that promote binding of a particular therapeutic agent. Each therapeutic agent has a particular shape, charge, and degree or level of hydrophobicity that may contribute to its ability to bind to the shell and or protein bound to the surface. The binding capacity as well as binding affinity between the therapeutic agent and the nanostructure may be regulated by modification to the therapeutic profile. For instance, a particular combination of lipids may provide an optimal surface for binding to a small molecule or protein. Positively charged head groups in the outer layer are shown to decrease the binding affinity, while negatively charged lipid head groups increase the binding affinity.

Examples of nanostructures that can be used in the methods are described herein are now described. The structure, nanostructure or nanoparticle (e.g., a synthetic structure or synthetic nanostructure) has a core and a shell surrounding the core. In embodiments in which the core is a nanostructure, the core includes a surface to which one or more components can be optionally attached. For instance, in some cases, core is a nanostructure surrounded by shell, which includes an inner surface and an outer surface. The shell may be formed, at least in part, of one or more components, such as a plurality of lipids, which may optionally associate with one another and/or with surface of the core. For example, components may be associated with the core by being covalently attached to the core, physiosorbed, chemisorbed, or attached to the core through ionic interactions, hydrophobic and/or hydrophilic interactions, electrostatic interactions, van der Waals interactions, or combinations thereof. In one particular embodiment, the core includes a gold nanostructure and the shell is attached to the core through a gold-thiol bond.

A number of therapeutic agents are typically associated with the shell of a nanostructure. For instance, at least 20 therapeutic agents may be associated per structure. In general at least 20-30, 20-40, 20-50, 25-30, 25-40, 25-50, 30-40, 30-50, 35-40, 35-50, 40-45, 40-50, 45-50, 50-100 or 30-100 therapeutic agents may be associated per structure.

Optionally, components can be crosslinked to one another. Crosslinking of components of a shell can, for example, allow the control of transport of species into the shell, or between an area exterior to the shell and an area interior of the shell. For example, relatively high amounts of crosslinking may allow certain small, but not large, molecules to pass into or through the shell, whereas relatively low or no crosslinking can allow larger molecules to pass into or through the shell. Additionally, the components forming the shell may be in the form of a monolayer or a multilayer, which can also facilitate or impede the transport or sequestering of molecules. In one exemplary embodiment, shell includes a lipid bilayer that is arranged to sequester cholesterol and/or control cholesterol efflux out of cells, as described herein.

It should be understood that a shell which surrounds a core need not completely surround the core, although such embodiments may be possible. For example, the shell may surround at least 50%, at least 60%, at least 70%, at least 80%, at least 90%, or at least 99% of the surface area of a core. In some cases, the shell substantially surrounds a core. In other cases, the shell completely surrounds a core. The components of the shell may be distributed evenly across a surface of the core in some cases, and unevenly in other cases. For example, the shell may include portions (e.g., holes) that do not include any material in some cases. If desired, the shell may be designed to allow penetration and/or transport of certain molecules and components into or out of the shell, but may prevent penetration and/or transport of other molecules and components into or out of the shell. The ability of certain molecules to penetrate and/or be transported into and/or across a shell may depend on, for example, the packing density of the components forming the shell and the chemical and physical properties of the components forming the shell. The shell may include one layer of material, or multilayers of materials in some embodiments.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the inner surface to the outer surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the inner surface to the outer surface of the shell). Such thicknesses may be determined prior to or after sequestration of molecules as described herein.

The shell of a structure described herein may comprise any suitable material, such as a hydrophobic material, a hydrophilic material, and/or an amphiphilic material. Although the shell may include one or more inorganic materials such as those listed above for the nanostructure core, in many embodiments the shell includes an organic material such as a lipid or certain polymers. The binding affinity of the nanoparticles may be further altered by including cholesterol (e.g., to modulate fluidity of the lipid monolayer or bilayer).

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer (e.g., lipid monolayer) or a multi-layer (e.g., a bilayer, lipid bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example, 1,2-Dipalmitoyl-sn-Glycero-3-Phosphothioethanol, phosphatidylcholine, phosphatidylglycerol, lecithin, β,γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol (DPPTE), and combinations thereof. In some cases, a shell (e.g., a bilayer) of a structure includes 50-200 natural or synthetic lipids or lipid analogs (e.g., phospholipids). For example, the shell may include less than about 500, less than about 400, less than about 300, less than about 200, or less than about 100 natural or synthetic lipids or lipid analogs (e.g., phospholipids), e.g., depending on the size of the structure.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E and K), glycerides (e.g., monoglycerides, diglycerides, triglycerides) can be used to form portions of a structure described herein.

A portion of a structure described herein such as a shell or a surface of a nanostructure may optionally include one or more alkyl groups, e.g., an alkane-, alkene-, or alkyne-containing species, that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The alkyl group may include any suitable end group, e.g., a thiol group, an amino group (e.g., an unsubstituted or substituted amine), an amide group, an imine group, a carboxyl group, or a sulfate group, which may, for example, allow attachment of a ligand to a nanostructure core directly or via a linker. For example, where inert metals are used to form a nanostructure core, the alkyl species may include a thiol group to form a metal-thiol bond. In some instances, the alkyl species includes at least a second end group. For example, the species may be bound to a hydrophilic moiety such as polyethylene glycol. In other embodiments, the second end group may be a reactive group that can covalently attach to another functional group. In some instances, the second end group can participate in a ligand/receptor interaction (e.g., biotin/streptavidin).

In some embodiments, the shell includes a polymer. For example, an amphiphilic polymer may be used. The polymer may be a diblock copolymer, a triblock copolymer, etc., e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, sytrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

Where a shell includes an amphiphilic material, the material can be arranged in any suitable manner with respect to the nanostructure core and/or with each other. For instance, the amphiphilic material may include a hydrophilic group that points towards the core and a hydrophobic group that extends away from the core, or, the amphiphilic material may include a hydrophobic group that points towards the core and a hydrophilic group that extends away from the core. Bilayers of each configuration can also be formed.

Core

The core of the nanostructure whether being a nanostructure core or a hollow core, may have any suitable shape and/or size. For instance, the core may be substantially spherical, non-spherical, oval, rod-shaped, pyramidal, cube-like, disk-shaped, wire-like, or irregularly shaped. The core (e.g., a nanostructure core or a hollow core) may have a largest cross-sectional dimension (or, sometimes, a smallest cross-section dimension) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, or less than or equal to about 5 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

The core may be formed of an inorganic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 wt %, 5 wt %, 10 wt %, 25 wt %, 50 wt %, 75 wt %, 90 wt %, or 99 wt %. In one embodiment, the core is formed of 100 wt % inorganic material. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nano structure includes or may be formed of one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen. In certain embodiments, the structure, nanostructure or nanoparticle core does not include a polymeric material (e.g., it is non-polymeric).

In some embodiments, the structure, nanostructure, or nanoparticle disclosed herein has 60-250 fold excess lipid to gold core. In some embodiments, the structure, nanostructure, or nanoparticle disclosed herein has 60-200, 60-150, 60-100, 60-75, 70-200, 70-150, 70-100, 70-75, 80-250, 80-200, 80-150, 80-100, 90-250, 90-200, 90-150, 90-100, 100-250, 100-200, 100-150, 62.5, 125, 187.5, or 250 fold excess lipid to the core (e.g., gold core).

Proteins

The structures described herein may also include one or more proteins, polypeptides and/or peptides (e.g., synthetic peptides, amphiphilic peptides). In one set of embodiments, the structures include proteins, polypeptides and/or peptides that can increase the rate of cholesterol transfer or the cholesterol-carrying capacity of the structures. The one or more proteins or peptides may be associated with the core (e.g., a surface of the core or embedded in the core), the shell (e.g., an inner and/or outer surface of the shell, and/or embedded in the shell), or both. Associations may include covalent or non-covalent interactions (e.g., hydrophobic and/or hydrophilic interactions, electrostatic interactions, van der Waals interactions).

An example of a suitable protein that may associate with a structure described herein is an apolipoprotein, such as apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Specifically, apo A1, apo A2, and apo E promote transfer of cholesterol and cholesteryl esters to the liver for metabolism and may be useful to include in structures described herein. Additionally or alternatively, a structure described herein may include one or more peptide analogues of an apolipoprotein, such as one described above. A structure may include any suitable number of, e.g., at least 1, 2, 3, 4, 5, 6, or 10, apolipoproteins or analogues thereof. In certain embodiments, a structure includes 1-6 apolipoproteins, similar to a naturally occurring HDL particle. Of course, other proteins (e.g., non-apolipoproteins) can also be included in structures described herein.

Optionally, one or more enzymes may also be associated with a structure described herein. For example, lecithin-cholesterol acyltransferase is an enzyme which converts free cholesterol into cholesteryl ester (a more hydrophobic form of cholesterol). In naturally-occurring lipoproteins (e.g., HDL and LDL), cholesteryl ester is sequestered into the core of the lipoprotein, and causes the lipoprotein to change from a disk shape to a spherical shape. Thus, structures described herein may include lecithin-cholesterol acyltransferase to mimic HDL and LDL structures. Other enzymes such as cholesteryl ester transfer protein (CETP) which transfers esterified cholesterol from HDL to LDL species may also be included.

It should be understood that the components described herein, such as the lipids, phospholipids, alkyl groups, polymers, proteins, polypeptides, peptides, enzymes, bioactive agents, nucleic acids, and species for targeting described above (which may be optional), may be associated with a structure in any suitable manner and with any suitable portion of the structure, e.g., the core, the shell, or both. For example, one or more such components may be associated with a surface of a core, an interior of a core, an inner surface of a shell, an outer surface of a shell, and/or embedded in a shell.

Additionally, the components described herein, such as the lipids, phospholipids, alkyl groups, polymers, proteins, polypeptides, peptides, enzymes, bioactive agents, nucleic acids, and species for targeting described above, may be associated with a structure described herein prior to administration to a subject or biological sample and/or after administration to a subject or biological sample. For example, in some cases a structure, nanostructure or nanoparticle (e.g., HDL nanoparticle) described herein includes a core and a shell which is administered in vivo or in vitro, and the structure has a greater therapeutic effect after sequestering one or more components (e.g., an apolipoprotein) from a subject or biological sample. That is, the structure may use natural components from the subject or biological sample to increase efficacy of the structure after it has been administered.

A variety of methods can be used to fabricate the structure, nanostructure or nanoparticle (e.g., HDL nanoparticle) described herein. Examples of methods are provided in International Patent Publication No. WO 2009/131704, filed Apr. 24, 2009 and entitled, “Nanostructures Suitable for Sequestering Cholesterol and Other Molecules”, which is incorporated herein by reference in its entirety for all purposes.

Cell

The structure, nanostructure or nanoparticle described herein may also be contacted with a cell. In some embodiments, the cell is a mammalian cell. For example, the genetic circuits described herein are contacted with human cells, primate cells (e.g., VERO cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, LNCaP (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSYSY human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, the structure, nanostructure or nanoparticle is contacted with a neutrophil cell. In other embodiments, the structure, nanostructure or nanoparticle is contacted with a muscle cell (e.g., human aortic smooth muscle cell [AoSMC]) or an endothelial cell (e.g., human aortic endothelial cell [HAEC]).

Pharmaceutical Compositions

As described herein, the inventive structures may be used in “pharmaceutical compositions” or “pharmaceutically acceptable” compositions, which comprise a therapeutically effective amount of one or more of the structures described herein, formulated together with one or more pharmaceutically acceptable carriers, additives, and/or diluents. The pharmaceutical compositions described herein may be useful for treating vascular diseases, angiogenesis, ischemia-reperfusion (e.g., ischemia reperfusion injury following organ transplantation) or other conditions. It should be understood that any suitable structures described herein can be used in such pharmaceutical compositions, including those described in connection with the figures. In some cases, the structures in a pharmaceutical composition have a nanostructure core comprising an inorganic material and a shell substantially surrounding and attached to the nanostructure core.

The pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The structures described herein may be orally administered, parenterally administered, subcutaneously administered, and/or intravenously administered. In certain embodiments, a structure or pharmaceutical preparation is administered orally. In other embodiments, the structure or pharmaceutical preparation is administered intravenously. Alternative routes of administration include sublingual, intramuscular, and transdermal administrations.

Pharmaceutical compositions described herein include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, from about 5% to about 70%, or from about 10% to about 30%.

The inventive compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a structure described herein as an active ingredient. An inventive structure may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered structure is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the structures described herein include pharmaceutically acceptable emulsions, microemulsions, solutions, dispersions, suspensions, syrups and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions described herein (e.g., for rectal or vaginal administration) may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the body and release the structures.

Dosage forms for the topical or transdermal administration of a structure described herein include powders, sprays, ointments, pastes, foams, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the structures described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a structure described herein to the body. Dissolving or dispersing the structure in the proper medium can make such dosage forms. Absorption enhancers can also be used to increase the flux of the structure across the skin. Either providing a rate controlling membrane or dispersing the structure in a polymer matrix or gel can control the rate of such flux.

Ophthalmic formulations, eye ointments, powders, solutions and the like, are also contemplated as being within the scope of this invention.

Pharmaceutical compositions described herein suitable for parenteral administration comprise one or more inventive structures in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Delivery systems suitable for use with structures and compositions described herein include time-release, delayed release, sustained release, or controlled release delivery systems, as described herein. Such systems may avoid repeated administrations of the structures in many cases, increasing convenience to the subject and the physician. Many types of release delivery systems are available and known to those of ordinary skill in the art. They include, for example, polymer based systems such as polylactic and/or polyglycolic acid, polyanhydrides, and polycaprolactone; nonpolymer systems that are lipid-based including sterols such as cholesterol, cholesterol esters, and fatty acids or neutral fats such as mono-, di- and triglycerides; hydrogel release systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The compositions may be as, for example, microspheres, hydrogels, polymeric reservoirs, cholesterol matrices, or polymeric systems. In some embodiments, the system may allow sustained or controlled release of the active compound to occur, for example, through control of the diffusion or erosion/degradation rate of the formulation. In addition, a pump-based hardware delivery system may be used in some embodiments. The structures and compositions described herein can also be combined (e.g., contained) with delivery devices such as syringes, pads, patches, tubes, films, MEMS-based devices, and implantable devices.

Use of a long-term release implant may be particularly suitable in some cases. “Long-term release,” as used herein, means that the implant is constructed and arranged to deliver therapeutic levels of the composition for at least about 30 or about 45 days, for at least about 60 or about 90 days, or even longer in some cases. Long-term release implants are well known to those of ordinary skill in the art, and include some of the release systems described above.

Injectable depot forms can be made by forming microencapsule matrices of the structures described herein in biodegradable polymers such as polylactide-polyglycolide. Depending on the ratio of structure to polymer, and the nature of the particular polymer employed, the rate of release of the structure can be controlled. Examples of other biodegradable polymers include poly(orthoesters) and poly(anhydrides).

When the structures described herein are administered as pharmaceuticals, to humans and animals, they can be given per se or as a pharmaceutical composition containing, for example, about 0.1% to about 99.5%, about 0.5% to about 90%, or the like, of structures in combination with a pharmaceutically acceptable carrier.

The administration may be localized (e.g., to a particular region, physiological system, tissue, organ, or cell type) or systemic, depending on the condition to be treated. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be useful for some treatments because of the convenience to the patient as well as the dosing schedule.

Regardless of the route of administration selected, the structures described herein, which may be used in a suitable hydrated form, and/or the inventive pharmaceutical compositions, are formulated into pharmaceutically-acceptable dosage forms by conventional methods known to those of skill in the art.

The compositions described herein may be given in dosages, e.g., at the maximum amount while avoiding or minimizing any potentially detrimental side effects. The compositions can be administered in effective amounts, alone or in a combinations with other compounds. For example, when treating cancer, a composition may include the structures described herein and a cocktail of other compounds that can be used to treat cancer. When treating conditions associated with abnormal lipid levels, a composition may include the structures described herein and other compounds that can be used to reduce lipid levels (e.g., cholesterol lowering agents).

The phrase “effective amount” as used herein means that amount of a material or composition comprising an inventive structure, nanostructure or nanoparticle which is effective for producing some desired biological effect. A “therapeutically effective amount” as used herein refers to an amount with a reasonable benefit/risk ratio applicable to any medical treatment. Accordingly, a therapeutically effective amount may, for example, prevent, minimize, or reverse disease progression associated with a disease or bodily condition, or donor graft (e.g., organ, tissue, etc.) rejection. Disease progression, disorder progression, or donor graft rejection can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The effective amount of any one or more structures described herein may be from about 10 ng/kg of body weight to about 1000 mg/kg of body weight, and the frequency of administration may range from once a day to once a month. However, other dosage amounts and frequencies also may be used as the invention is not limited in this respect. A subject may be administered one or more structure described herein in an amount effective to treat one or more diseases or bodily conditions described herein.

An effective amount may depend on the particular condition to be treated. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

The compositions containing an effective amount can be administered for prophylactic or therapeutic treatments. In prophylactic applications, compositions can be administered to a patient with a clinically determined predisposition or increased susceptibility to development of a NO deficiency disorder, cardiovascular diseases, hyperproliferative diseases (e.g., cancer), inflammatory diseases, diabetes, dyslipidemia, and other pathological conditions associated with oxidative stress, an imbalance in redox homeostasis, immune dysfunction, and/or endothelia dysfunction. Compositions of the invention can be administered to the patient (e.g., a human) in an amount sufficient to delay, reduce, or preferably prevent the onset of the clinical disease. In therapeutic applications, compositions are administered to a patient (e.g., a human) already suffering from a NO deficiency disorder, cardiovascular disease, hyperproliferative diseases (e.g., cancer), an inflammatory disease, diabetes, dyslipidemia, and other pathological conditions associated with oxidative stress, an imbalance in redox homeostasis, immune dysfunction, and/or endothelial dysfunction, in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or a medical condition. For example, in the treatment of a NO deficiency disorder, cardiovascular disease, hyperproliferative diseases (e.g., cancer), an inflammatory disease, diabetes, dyslipidemia, and other pathological conditions associated with oxidative stress, an imbalance in redox homeostasis, immune dysfunction, and/or endothelia dysfunction, an agent or composition which decreases, prevents, delays, suppresses, or arrests any symptom of the disease or condition would be therapeutically effective. A therapeutically effective amount of an agent or composition is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular inventive structure employed, the route of administration, the time of administration, the rate of excretion or metabolism of the particular structure being employed, the duration of the treatment, other drugs, compounds and/or materials used in combination with the particular structure employed, the age, sex, weight, condition, general health and prior medical history of the patient being treated, and like factors well known in the medical arts.

Subject

As used herein, a “subject” or a “patient” refers to any mammal (e.g., a human), for example, a mammal that may be susceptible to a disease or bodily condition such as a disease or bodily condition that is, for instance, a vascular condition, disease or disorder (e.g., ischemia reperfusion injury after organ transplant). Examples of subjects or patients include a human, a non-human primate, a cow, a horse, a pig, a sheep, a goat, a dog, a cat or a rodent such as a mouse, a rat, a hamster, or a guinea pig. A subject may be a subject diagnosed with a certain disease or bodily condition or otherwise known to have a disease or bodily condition. In some embodiments, a subject may be diagnosed as, or known to be, at risk of developing a disease or bodily condition. In some embodiments, a subject may be diagnosed with, or otherwise known to have, for instance, a vascular condition, disease or disorder, as described herein. In certain embodiments, a subject may be selected for treatment on the basis of a known disease or bodily condition in the subject. In some embodiments, a subject may be selected for treatment on the basis of a suspected disease or bodily condition in the subject. In some embodiments, the composition may be administered to prevent the development of a disease or bodily condition. However, in some embodiments, the presence of an existing disease or bodily condition may be suspected, but not yet identified, and a composition of the present invention may be administered to diagnose or prevent further development of the disease or bodily condition.

A “biological sample,” as used herein, is any cell, body tissue, or body fluid sample obtained from a subject. Non-limiting examples of body fluids include, for example, lymph, saliva, blood, urine, and the like. Samples of tissue and/or cells for use in the various methods described herein can be obtained through standard methods including, but not limited to, tissue biopsy, including punch biopsy and cell scraping, needle biopsy; or collection of blood or other bodily fluids by aspiration or other suitable methods.

The function and advantage of these and other embodiments will be more fully understood from the examples below. The following examples are intended to illustrate the benefits of the present invention, but do not exemplify the full scope of the invention. Accordingly, it will be understood that the example section is not meant to limit the scope of the invention.

### EXAMPLES

### Example 1

### Methods

DPPTE is dissolved in 100% ethanol, then diluted with water to 40% ethanol (60% water). HCl is added to adjust the pH to ˜3. Sodium nitrite is dissolved in water, diluted to match the concentration of the DPPTE, then added to the DPPTE solution (20% ethanol final concentration). The metal chelator DTPA is added at a final concentration of 50 uM. The solution is vortexed and incubated at room temperature in the dark for ˜1 hour. The reaction is stopped by neutralizing the acid (pH=7) and the modified phospholipid is stored at −20° C. The SNO DPPTE is purified by HPLC using a methanol:water gradient, lyophilized and dissolved in 100% ethanol. NO HDL NPs are synthesized using the same protocol as HDL NPs. 5 nm citrate stabilized gold nanoparticles are surface functionalized by addition of 5 fold molar excess of apolipoprotein A1 for 1 hour at room temperature, followed by addition of 250-fold molar excess of the phospholipid PDP PE (disulfide containing phospholipid) and 250-fold molar excess SNO DPPTE. The nanoparticles are rocked over night at room temperature, then purified using tangential flow filtration (TFF).

### Example 2: Synthesis of High-Density Lipoprotein-Like (HDL) Nanoparticles for NO Delivery with Application to Ischemia/Reperfusion Injury

Ischemia/reperfusion injury (IRI), defined as a period of hypoxia or anoxia followed by reintroduction of oxygen, plays a critical role in a number of different pathologies, from myocardial infarction and stroke, to damage of transplanted organs and tissues¹⁻⁵. In the case of transplantation, donor organs experience two distinct phases of ischemia: an acute period of warm ischemia, from the time of complete occlusion of blood flow (i.e. cross-clamp) to organ harvest, and then a more prolonged period of cold ischemia, where the organ is perfused with cold preservation solution, transported, and eventually transplanted into the recipient^{6,7}. Following transplantation, the donor organ undergoes reperfusion, where the sudden influx of oxygen exacerbates ischemic damage, which can lead to delayed graft function, among other things. With the scarcity of donor organs, maximizing graft function is critical, especially those from marginal donors.

IRI is one of the major contributors to delayed graft function, a relatively common complication that presents immediately post transplantation and factors in determining the long-term outcome of the transplanted organ⁸⁻¹⁰. During the process of IRI in allogeneic kidney transplants, the innate and adaptive immune systems are activated, leading to infiltration of the kidney graft by host immune cells^{6,11-13}. The acute inflammatory response increases the immunogenicity of the transplanted graft, potentially leading to graft rejection. Numerous strategies exist to mitigate IRI, including, strict selection of the donor, minimized cold ischemia time, and administration of anti-inflammatory drugs⁷.

NO is a gaseous molecule with potent biological effects. NO is a potent vasodilator and mediates intracellular signaling¹⁴. Altered NO levels have been implicated in a variety of disorders, including sickle cell disease, erectile dysfunction, rheumatoid arthritis, atherosclerosis, and ischemia/reperfusion injury. NO plays a significant role in protecting cells from IRI; however, prolonged periods of ischemia leads to decreased expression and activity of endothelial NOS in endothelial cells¹⁵. Restoration of NO levels, through delivery of exogenous NO, may ameliorate IRI and improve graft function.

Due to the fact that NO exists as a free radical gas, its half-life in biological systems is extremely short, on the order of milliseconds or less. Most NO delivery methods utilize an NO donor, such as a diazeniumdiolate, or involve the use of inhaled NO gas¹⁶. However, these compounds suffer from short half-lives, and unfavorable biodistribution patterns. Several nanoparticles have been developed as delivery platforms for NO¹⁷. While some metal/metal oxide nanoparticles have been developed to deliver NO¹⁸⁻²⁰, the majority of research has focused on silica nanoparticles. Generally speaking, these silica nanoparticles are functionalized with diazeniumdiolates as a method to release NO, and have been shown to decrease blood pressure, increase vasodialation and ameliorate hemoglobin-induced vasoconstriction in hamsters^{21,22}. With respect to ischemia/reperfusion injury, conjugation of the NO donor SNAP (S-nitroso-N-acetyl-D,L-penicillamine) to a dendrimer nanoparticulate scaffold reduced the size of infarction injury in explanted rat hearts²³. While these results are promising, significant limitations of silica and dendrimeric nanoparticles, including stability in aqueous solutions, release of NO prior to injection, relatively poor stability of diazeniumdiolates, and a lack of targeting, render these nanostructures poorly suited for in vivo applications.

The synthesis and characterization of high-density lipoprotein-like nanoparticles (HDL NPs), which mimic the size, shape, surface composition, and some functions of natural HDLs, have been previously described.²⁴⁻²⁶ HDL NPs are composed of a 5 nm gold nanoparticle core, surface functionalized with the HDL-defining apolipoproteinA-I, and a phospholipid bilayer. Natural HDL and HDL NPs inherently target cell types critical to IRI, including endothelial cells and immune cells. The hypothesis is that incorporation of an S-nitrosylated phospholipid (SNO-PL) into the bilayer of HDL NPs would allow for the delivery of NO both in vitro and in vivo.

### Results

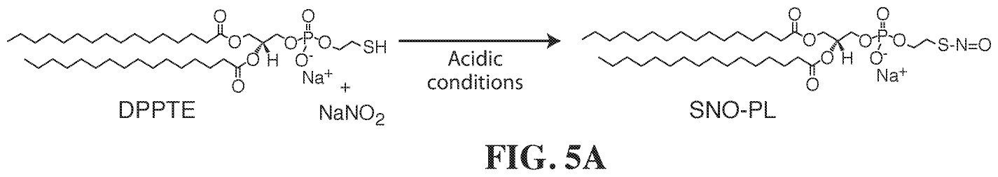

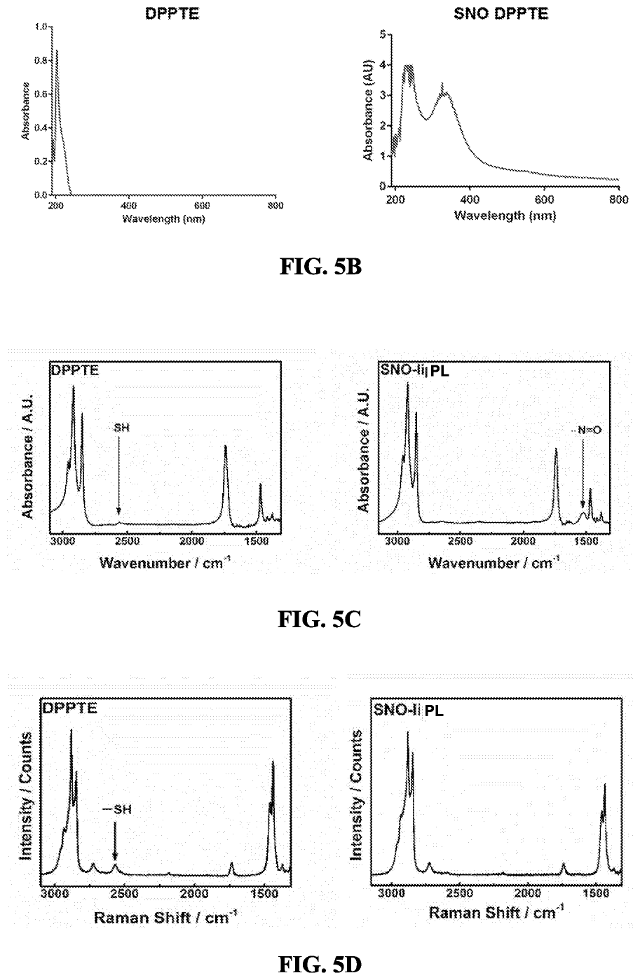

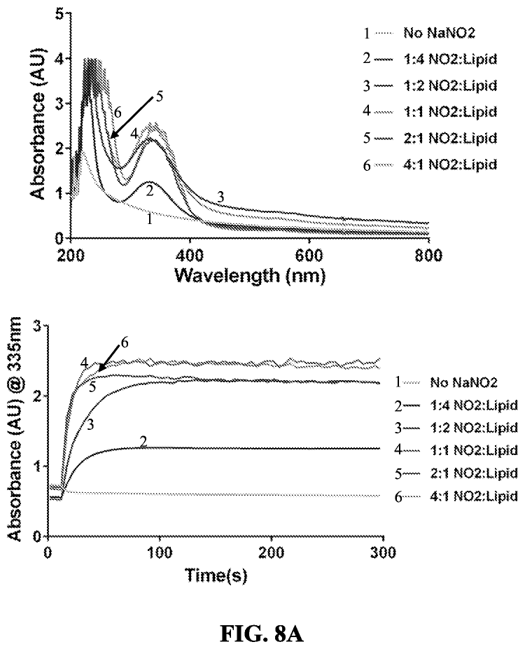

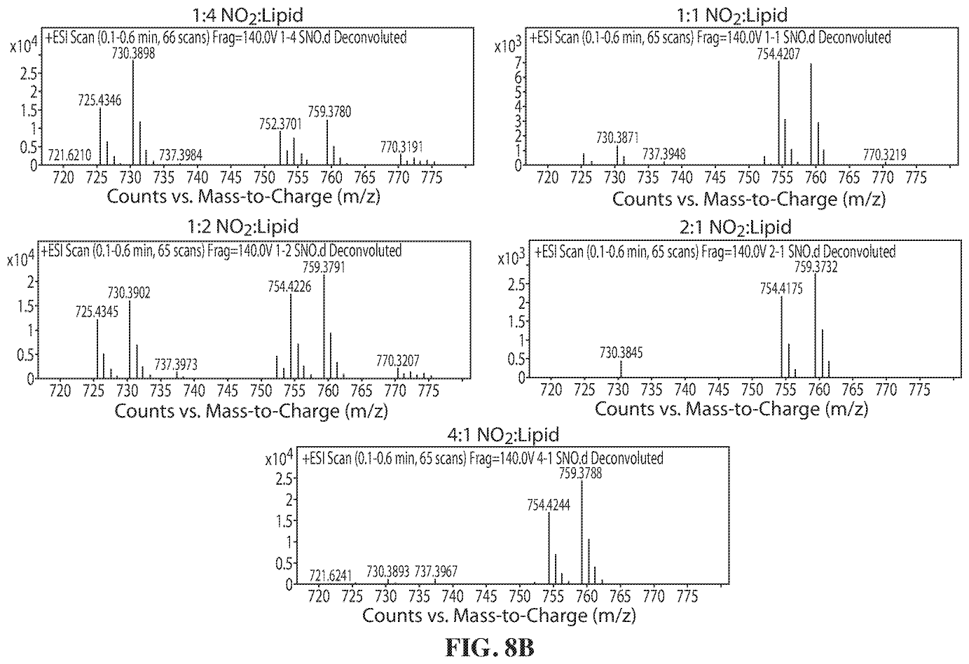

The commercially available, thiol-containing phospholipid (PL) 1,2-dipalmitoyl-sn-glycero-3-phosphothioethanol (DPPTE) was employed. This PL was S-nitrosylated by addition of an equimolar quantity of NaNO₂ under acidic (pH=3) conditions (FIG. 5A). The —S—N═O moiety has an absorbance maximum at 335 nm, which allows for reaction monitoring by UV/Vis spectroscopy. Addition of NaNO₂ (FIG. 5B) to an equimolar concentration of the phospholipid resulted in complete and rapid conversion of DPPTE to SNO-PL (FIG. 5C, FIG. 8A). Altering the ratio of NaNO₂ to DPPTE did not result in increased S-nitrosylation or faster reaction kinetics (FIG. 8B). FTIR and Raman spectroscopy of SNO-PL and DPPTE further confirmed the transformation of the —S-H group to an —S—N═O moiety (FIG. 5D, FIG. 8B).

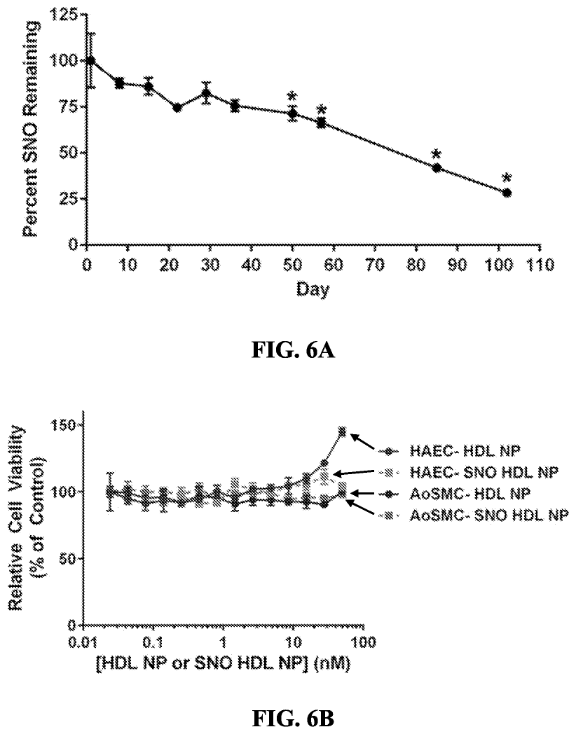

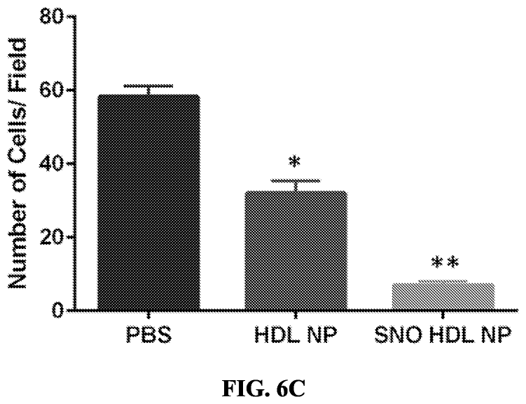

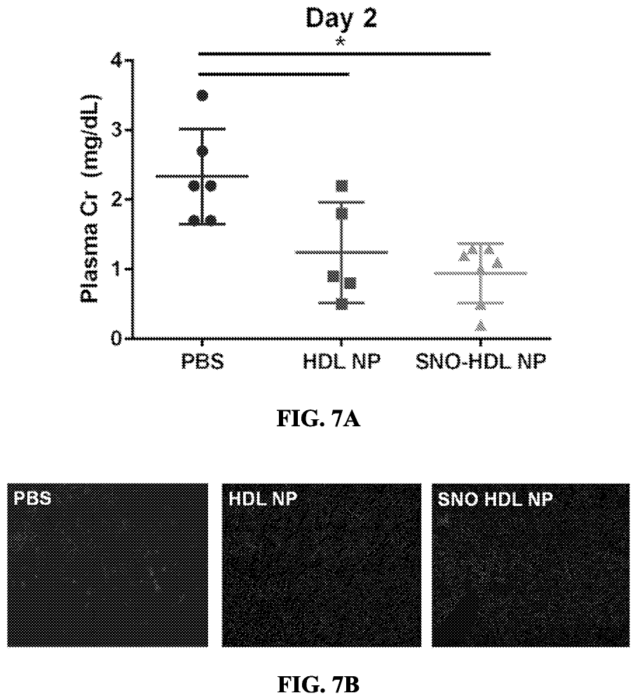

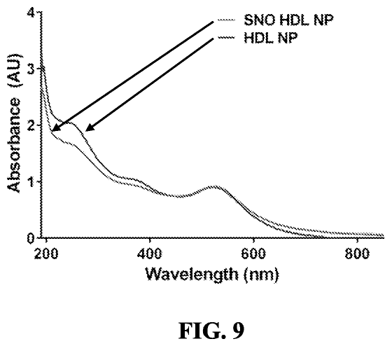

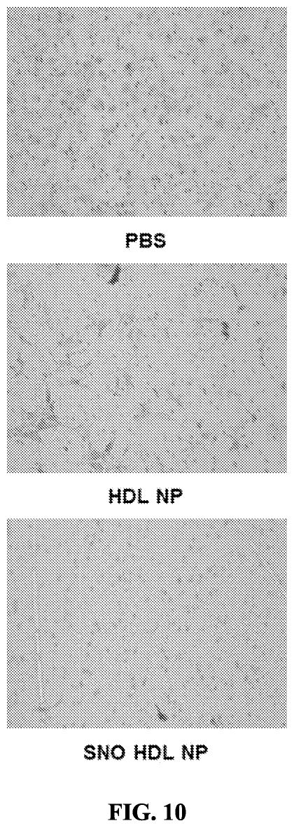

SNO HDL NP synthesis was carried out using standard protocols, with gold colloid, apoA-I and phospholipids in a 20% ethanol/80% water (v/v) solution. The conjugates were purified by tangential flow filtration, as previously described²⁴⁻²⁶. UV/Vis spectroscopy of SNO HDL NPs was similar to spectra for HDL NPs, with a local maximum at ˜520 nm corresponding to the surface plasmon resonance of the gold nanoparticle. Due to signal from the gold nanoparticle and apoA-I, no peak at 335 nm was detected in the SNO HDL NPs (FIG. 9). Chemiluminescent detection, using a Sievers Nitric Oxide Analyzer (NOA), and a solution of I₃⁻ in glacial acetic acid²⁷, demonstrated the presence of the SNO groups on SNO HDL NPs (FIG. 6A). The SNO groups were stable on the SNO HDL NPs, when stored at 4° C., with 71.4%±3.9% SNO remaining after 50 days, and 28.4%±1.3% after 100 days (FIG. 6A). SNO HDL NP and HDL NP toxicity towards human aortic endothelial cells (HAEC) and human aortic smooth muscle cells (AoSMC), two of the expected cell types to interact with the nanoparticles, was quantified using the MTS assay. Both nanoparticle constructs were non-toxic in the HAECs and AoSMCs (FIG. 6B). To verify that the SNO HDL NPs could successfully deliver a physiologically relevant dose of NO, the ability of the SNO HDL NPs to inhibit migration of aortic smooth muscle cells was tested. NO has been shown to inhibit the migration of smooth muscle cells, both in vitro and in vivo. SNO HDL NPs significantly inhibited AoSMC migration in a transwell migration assay (FIG. 6C; FIGS. 7A-7B). Interestingly, the HDL NP construct partially inhibited AoSMC migration, suggesting that the inherent functionality of the HDL NP beyond the ability to deliver NO.

To investigate the ability of the SNO HDL NPs and HDL NPs to ameliorate IRI in kidney transplantation, a mouse kidney transplant model was utilized. Kidneys were harvested from donor mice, placed on ice for 4 hours, and then transplanted to a recipient mouse that has undergone a bilateral nephrectomy, leaving the transplanted kidney graft as the only functional kidney remaining. Donors were treated with nanoparticles prior to organ harvest, the organ perfused with nanoparticles during cold ischemia incubation, and the recipient mouse treated with nanoparticles immediately following surgery and again 24 hours later. Plasma creatinine was measured on day 2. HDL NP and SNO HDL NP both decreased plasma creatinine levels compared to controls (2.333±0.683 mg/dL for PBS treated v. 1.240±0.723 for HDL NP treated v. 0.943±0.428 for SNO HDL NP treated; p<0.05 v. PBS treated v. nanoparticles; FIG. 7A).

### Discussion

Interestingly, the HDL NP construct itself ameliorated some of the IRI damage, suggesting that HDL NPs have an inherent ability to protect kidney tissue from IRI. It should be noted that in this transplant model, plasma creatinine levels returned to baseline values around day 14, a time line that is far shorter than in human kidney transplant recipients.

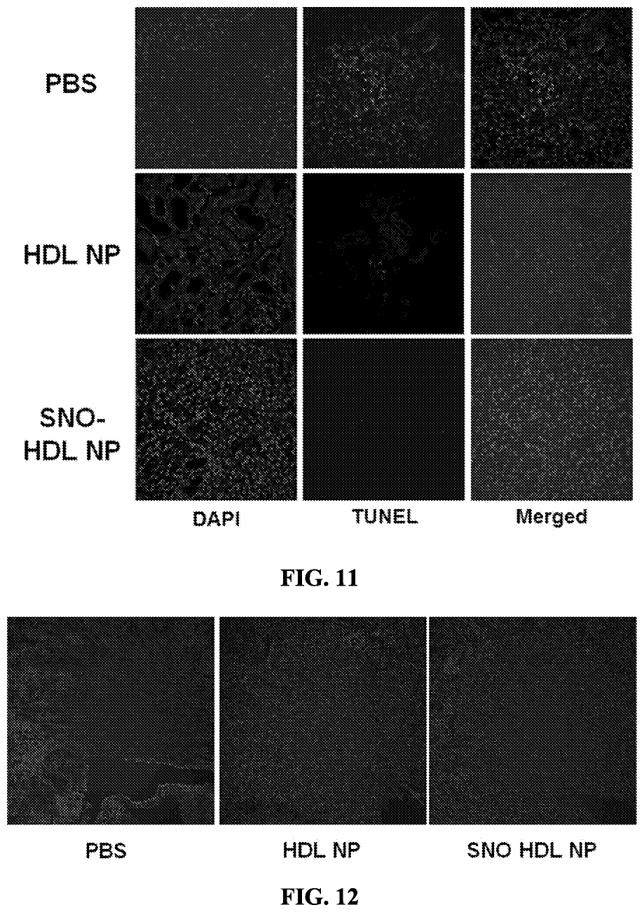

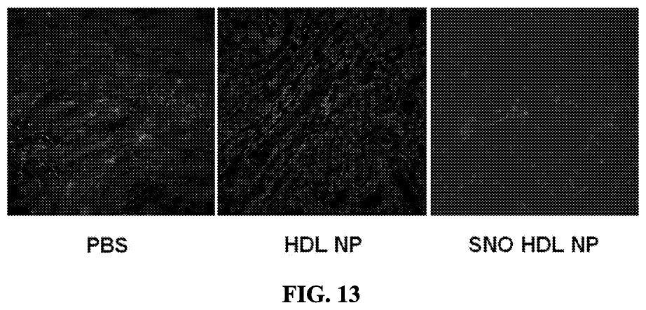

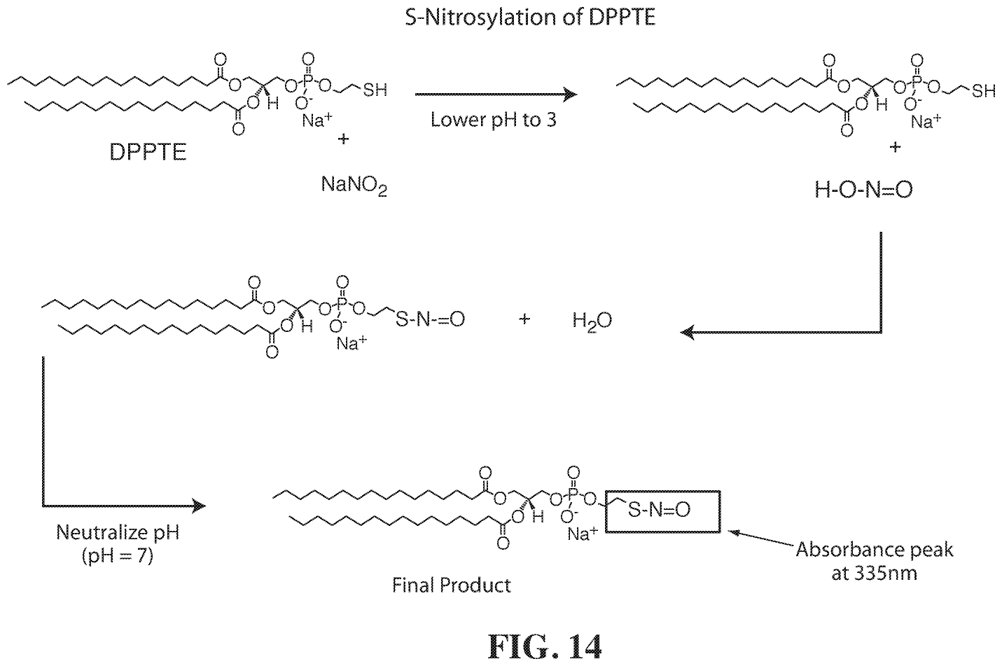

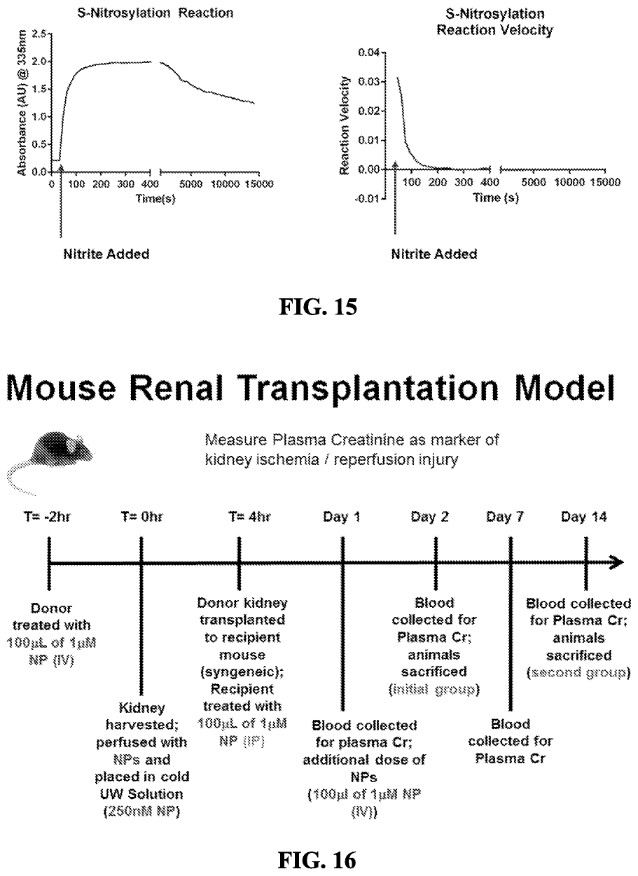

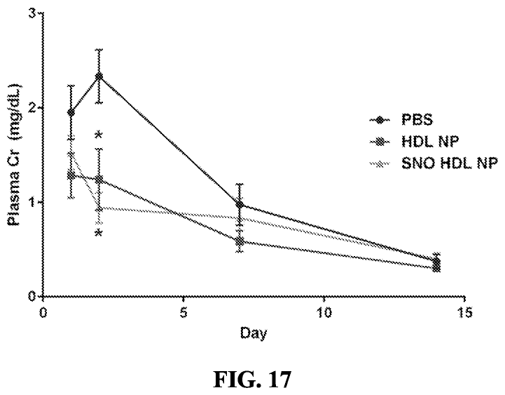

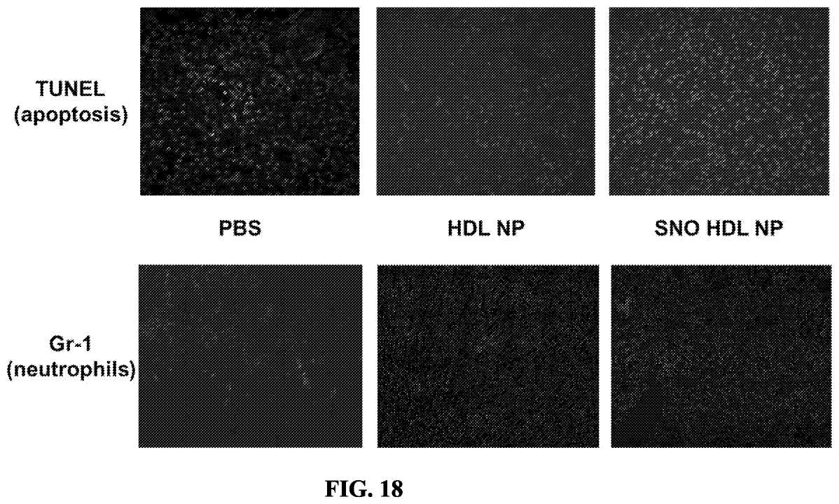

Immunocytochemical staining for apoptosis (TUNEL) and proliferation (Ki67) demonstrated that HDL NP and SNO HDL NP both decreased the number of apoptotic cells and increased the number of proliferating cells (FIG. 11). Macrophage infiltration in the grafts was similar across all treatment groups (FIG. 12). Infiltration by neutrophils, visualized by staining the kidney grafts for Gr-1, was reduced in SNO HDL NPs compared to HDL NP and PBS controls (FIG. 7B). These data suggest that the HDL NP construct itself acts to prevent apoptosis and induce proliferation in renal cells, while the NO delivered by the SNO HDL NP limited infiltration of the transplanted kidney by neutrophils.

In conclusion, the high density lipoprotein-like nanoparticles can be successfully loaded with an S-nitrosylated phospholipid, resulting in a NO-releasing HDL NP that is stable, non-toxic, and capable of delivering a physiologically relevant dose of NO both in vitro and in an in vivo model of kidney transplantation.

### Materials and Methods

### Preparation of S-nitrosylated 1,2-dipalmitoyl-sn-glycero-3-phosphothioethanol (SNO-PL)

The commercially available thiol containing phospholipid DPPTE was S-nitrosylated through addition of sodium nitrite under acidic conditions. DPPTE was reconstituted in 100% ethanol to a concentration of 25 mM then diluted with water to a final concentration of 5 mM DPPTE, in 20% ethanol/80% water. The pH of the solution was lowered to 3 by addition of HCl. Pentatonic acid (DPTA) was added at a final concentration of 50 uM, to chelate any heavy metal ions that may be present. The S—N═O group is particularly susceptible to degradation by heavy metals such as copper and zinc, necessitating the strong chelating agent DPTA. Finally, sodium nitrite was added to the solution and the reaction tracked using UV/Vis spectroscopy. The S—N═O group has an absorbance maximum at 335 nm, and the accumulation of the S-nitrosylated product can be monitored over time. The typical ratio of DPPTE to sodium nitrite was 1:1 unless otherwise stated. UV/Vis spectroscopy was used both to characterize the end product as well as monitor reaction progression over time.

Characterization of SNO-PL.

Following synthesis, the SNO-PL was characterized by mass spectroscopy, FTIR, Raman spectroscopy, and UV/Vis spectroscopy. For mass spectroscopy, a small sample of each reaction (e. g. different ratio of phospholipid to sodium nitrite) was run on the mass spectrometer. FTIR and Raman spectroscopy was also performed. SNO-PL was first dried under nitrogen prior to analysis.

High Density Lipoprotein-Like Nanoparticle Synthesis.

HDL NP synthesis was carried out as previously described in patents, such as U.S. Pat. No. 8,323,686. Briefly, 5 nm citrate stabilized gold nanoparticles were surface functionalized with a 5-fold molar excess of apolipoprotein A1 and a phospholipid bilayer, with each phospholipid added at a 250-fold molar excess relative to the gold nanoparticle concentration. The disulfide containing phospholipid PDP PE was used as the inner phospholipid in all syntheses. The outer phospholipid of the HDL NPs was a combination of DPPC and the SNO-PL. Following an overnight incubation, the HDL NPs were then subjected to tangential flow filtration. The concentration of the HDL NPs was determined using Beer's law and UV/Vis spectroscopy. The various constructs were analyzed for their size (dynamic light scattering; DLS) and surface charge (zeta potential), using the Malvern zetasizer.

NO content and long-term stability of the SNO group was assayed using the Sievers Nitric Oxide analyzer (NOA) and the tri-iodine method, described previously. Briefly, a solution of iodine and iodide was mixed with glacial acetic acid and loaded into the NOA. SNO HDL NP samples were injected into the solution, which then released any still bound nitric oxide into the instrument, where it combined with oxygen to produce a chemiluminescent signal. Samples of the SNO HDL NPs were taken over time, and the percentage of SNO groups remaining reported here.

Toxicity.

The MTS assay was used to quantify the toxicity of SNO HDL NPs and HDL NPs on human aortic endothelial cells and human arterial smooth muscle cells. Cells were plated at 1*10⁵ cells/ml into 96 well plates, and were treated with HDL NPs or SNO HDL NPs for 48 hours prior to addition of the MTS reagent. A Biotek Synergy 2 plate reader was used to measure the absorbance at 490 nm prior to MTS reagent addition, at time=0, and at time=120 minutes. Percent viability was calculated by subtracting the time=0 values from the time=120 values, then standardizing the resultant values to the PBS control (set to 100%).

Transwell Migration Assay.

AoSMCs were resuspended at a concentration of 1*10⁶ cells/ml, and 100 μl of cells was added to the interior of an 8 μm pore size transwell insert placed in a 24 well plate. The cells were incubated for 10 minutes to allow for attachment, then 600 μl of culture media+treatment was added. HDL NPs and SNO HDL NPs were added at a final concentration of 50 nM. The cells were incubated for 4 hours, then washed twice with PBS, and fixed with 100% ethanol. Following fixation, the cells were stained with crystal violet and the number of cells migrating through the insert calculated by averaging 10 fields per replicate.

Murine Kidney Transplantation Model.

Donor C57/B16 mice were injected with 100 μl of PBS, 1 μM HDL NPs or 1 μM SNO HDL NPs 2 hours prior to harvesting of the donor kidney. The kidney was resected, along with a portion of the aorta and inferior vena cava, perfused with a cold solution of 250 nM HDL NP or SNO HDL NP in University of Wisconsin (UW) solution. The donor organ was transferred to 4° C. for 4 hours prior to transplantation into the recipient C57/B16 mouse. The recipient mouse underwent a bilateral nephrectomy, with the first native kidney removed prior to transplantation and the second native kidney removed following transplantation. The transplanted kidney is connected to the vasculature using the aorta and vena cava segments retained from the donor. Following transplantation, mice were treated with either 100 μl of PBS, 1 μM HDL NP or 1 μM SNO HDL NP intraperitoneally. The following day, the recipients received an additional dose, this time via tail vein. Blood was collected on Day 2 and analyzed for plasma creatinine level. The transplanted organs were also resected, fixed with formalin, embedded in O.C.T. to investigate infiltration of the grafts by immune cells (e.g Gr-1), apoptosis (TUNEL staining), proliferation (Ki67) and gross histology (H&E).

Fluorescent Microscopy.

The Nikon AlR GaAsP confocal fluorescent microscope was used to image immunocytochemical stained kidney sections.

### REFERENCES

(1) Bagheri, F.; Khori, V.; Alizadeh, A. M.; Khalighfard, S.; Khodayari, S.; Khodayari, H. Life sciences 2016, 165, 43.
(2) Bonventre, J. V.; Yang, L. The Journal of clinical investigation 2011, 121, 4210.
(3) Munshi, R.; Hsu, C.; Himmelfarb, J. BMC medicine 2011, 9, 11.
(4) Piper, H. M.; Meuter, K.; Schafer, C. The Annals of thoracic surgery 2003, 75, S644.
(5) Yellon, D. M.; Hausenloy, D. J. The New England journal of medicine 2007, 357, 1121.
(6) Eltzschig, H. K.; Eckle, T. Nature medicine 2011, 17, 1391.
(7) Salvadori, M.; Rosso, G.; Bertoni, E. World journal of transplantation 2015, 5, 52.
(8) Cooper, J. E.; Wiseman, A. C. Current opinion in nephrology and hypertension 2013, 22, 698.
(9) Ortiz, F.; Paavonen, T.; Tornroth, T.; Koskinen, P.; Finne, P.; Salmela, K.; Kyllonen, L.; Gronhagen-Riska, C.; Honkanen, E. Journal of the American Society of Nephrology: JASN 2005, 16, 817.
(10) Ponticelli, C. Nephrology, dialysis, transplantation: official publication of the European Dialysis and Transplant Association—European Renal Association 2014, 29, 1134.
(11) Denecke, C.; Tullius, S. G. Progres en urologie: journal de l'Association francaise d'urologie et de la Societe francaise d'urologie 2014, 24 Suppl 1, S13.
(12) Erpicum, P.; Detry, O.; Weekers, L.; Bonvoisin, C.; Lechanteur, C.; Briquet, A.; Beguin, Y.; Krzesinski, J. M.; Jouret, F. Nephrology, dialysis, transplantation: official publication of the European Dialysis and Transplant Association—European Renal Association 2014, 29, 1487.
(13) Jang, H. R.; Rabb, H. Nature reviews. Nephrology 2015,11, 88.
(14) Schulz, R.; Kelm, M.; Heusch, G. Cardiovascular research 2004, 61, 402.
(15) Giraldez, R. R.; Panda, A.; Xia, Y.; Sanders, S. P.; Zweier, J. L. The Journal of biological chemistry 1997, 272, 21420.
(16) Deshpande, S. R.; Satyanarayana, K.; Rao, M. N.; Pal, K. V. Indian journal of pharmaceutical sciences 2012, 74, 487.
(17) Quinn, J. F.; Whittaker, M. R.; Davis, T. P. Journal of controlled release: official journal of the Controlled Release Society 2015, 205, 190.
(18) Polizzi, M. A.; Stasko, N. A.; Schoenfisch, M. H. Langmuir: the ACS journal of surfaces and colloids 2007, 23, 4938.
(19) Rothrock, A. R.; Donkers, R. L.; Schoenfisch, M. H. J Am Chem Soc 2005, 127, 9362.
(20) Zhang, X. F.; Mansouri, S.; Mbeh, D. A.; Yahia, L.; Sacher, E.; Veres, T. Langmuir: the ACS journal of surfaces and colloids 2012, 28, 12879.
(21) Cabrales, P.; Han, G.; Nacharaju, P.; Friedman, A. J.; Friedman, J. M. American journal of physiology. Heart and circulatory physiology 2011, 300, H49.
(22) Cabrales, P.; Han, G.; Roche, C.; Nacharaju, P.; Friedman, A. J.; Friedman, J. M. Free radical biology & medicine 2010, 49, 530.
(23) Johnson, T. A.; Stasko, N. A.; Matthews, J. L.; Cascio, W. E.; Holmuhamedov, E. L.; Johnson, C. B.; Schoenfisch, M. H. Nitric oxide: biology and chemistry/official journal of the Nitric Oxide Society 2010, 22, 30.
(24) Luthi, A. J.; Lyssenko, N. N.; Quach, D.; McMahon, K. M.; Millar, J. S.; Vickers, K. C.; Rader, D. J.; Phillips, M. C.; Mirkin, C. A.; Thaxton, C. S. Journal of lipid research 2015.
(25) Luthi, A. J.; Zhang, H.; Kim, D.; Giljohann, D. A.; Mirkin, C. A.; Thaxton, C. S. ACS Nano 2012, 6, 276.
(26) Thaxton, C. S.; Daniel, W. L.; Giljohann, D. A.; Thomas, A. D.; Mirkin, C. A. J Am Chem Soc 2009, 131, 1384.
(27) MacArthur, P. H.; Shiva, S.; Gladwin, M. T. Journal of chromatography. B, Analytical technologies in the biomedical and life sciences 2007, 851, 93.

All publications, patents and sequence database entries mentioned in the specification herein are hereby incorporated by reference in their entirety as if each individual publication or patent was specifically and individually indicated to be incorporated by reference. In case of conflict, the present application, including any definitions herein, will control.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A high density lipoprotein (HDL) nanoparticle comprising:
a core; a shell surrounding and attached to the nanostructure core, wherein the shell is comprised of apolipoprotein and reservoir molecules comprising nitric oxide (NO).

2. The HDL nanoparticle of claim 1, wherein the reservoir molecule is a lipid.

3. The HDL nanoparticle of claim 1, wherein the reservoir molecule is a phospholipid.

4. The HDL nanoparticle of claim 1, wherein the reservoir molecule is a modified phospholipid.

5. The HDL nanoparticle of claim 2, wherein the lipid contains an NO donating group.

6. The HDL nanoparticle of claim 1, wherein the reservoir molecule is a S-Nitrosylated lipid.

7. The HDL nanoparticle of claim 1, wherein the reservoir molecule is S-Nitrosylated 1,2-dipalmitoyl-sn-glycero-3-phosphothioethanol (DPPTE).

8-11. (canceled)

12. The HDL nanoparticle of claim 2, wherein the HDL nanoparticle has 60-250 fold excess lipid to gold core.

13-16. (canceled)

17. A method for delivering NO to a subject comprising:
administering to the subject the HDL nanoparticle of claim 1 to deliver NO to a cell in the subject.

18-33. (canceled)

34. A method for reducing migration of a cell, comprising contacting the cell with an effective amount of the structure comprising a core; a shell surrounding and attached to the nanostructure core, wherein the shell is comprised of apolipoprotein and reservoir molecules comprising nitric oxide (NO) to reduce migration of the cell relative to a cell without exposure to the structure.

35. The method of claim 34, wherein the cell is a neutrophil cell.

36. A method for treating a nitric oxide (NO)-mediated disorder comprising:
administering to a subject having a NO-mediated disorder an effective amount of a nanostructure comprising a core, a shell surrounding and attached to the core, wherein the shell is comprised of reservoir molecules comprising NO to deliver NO to a cell of the subject and treat the NO-mediated disorder.

37. The method of claim 36, wherein the reservoir molecule is a lipid.

38. The method of claim 36 or claim 37, wherein the reservoir molecule is a phospholipid.

39-50. (canceled)

51. The method of claim 36, wherein the NO-mediated disorder is angiogenesis.

52. The method of claim 36, wherein the NO-mediated disorder is ischemia-reperfusion injury.

53. The method of claim 36, wherein the NO-mediated disorder is ischemia-reperfusion injury following organ transplantation.

54. The method of claim 53, wherein the organ is a kidney.

55-56. (canceled)

57. A method for transplanting a donor organ in a recipient subject comprising:
harvesting a donor organ;
contacting the donor organ with a nanostructure comprising a core, a shell surrounding and attached to the core, wherein the shell is comprised of reservoir molecules comprising nitric oxide (NO); and
transplanting the donor organ into a recipient subject, wherein the nanostructure reduces the risk of rejection of the donor organ relative to the risk of a donor organ transplanted without exposure to the nanostructure.

58. The method of claim 57, wherein the nanostructure is administered to the recipient subject after the donor organ is transplanted.

59. The method of claim 57, wherein the nanostructure is administered to the donor before the donor organ is harvested.

60. The method of claim 57, wherein the donor organ is contacted with the nanostructure after the donor organ is harvested and before the donor organ is transplanted.

61. The method of claim 57, wherein the nanostructure is administered to the recipient subject immediately after the donor organ is transplanted.

62. The method of claim 57, further comprising administering to the recipient subject the nanostructure 24 hours after the donor organ is transplanted.

63. The method of claim 57, wherein the nanostructure reduces the levels of plasma creatine in the recipient subject relative to a recipient subject that received a transplanted donor organ without exposure to the nanostructure.

64. The method of claim 57, wherein the nanostructure reduces apoptosis of a cell in the donor organ relative to a cell in a donor organ transplanted without exposure to the nanostructure.

65. The method of claim 57, wherein the structure increases proliferation of a cell in the donor organ relative to a cell in a donor organ transplanted without exposure to the nanostructure.

66. The method of claim 57, wherein the transplanted organ is a kidney.

67. The method of claim 57, wherein the recipient subject is a mammal.

68. The method of claim 57, wherein the recipient subject is a human.

69. The method of claim 57, wherein the donor subject is a mammal.

70. The method of claim 57, wherein the donor subject is a human.

71-86. (canceled)
